SEQUENCE LISTING (17 sequences)

SEQ ID NO: 1 - protein, 397 aa, Aphis gossypii
MLGTQSRFVQRPQNKPDTMTELADMFYTLVFGDTEFSVPRRYVDLVPKGLGAQGMVVAAMDTITQQSVAIKKLSRPFQNVTHAKRAYREFKLMKLVNHKNIIGLLNAFTPQRTLDDFQDVYLVMELMDANLCQVIQMDLDHERMSYLLYQMLCGIKHLHSAGIIHRDLKPSNIVVKSDCTLKILDFGLARTAGTTFMMTPYVVTRYYRAPEVILGMGYKENVDIWSVGCIMGEMIRGGVLFPGTDHIDQWNKIIEQLGTPSQEFMRRLQPTVRNYVENRPRYPGYSFDRLFPDVLFPSDSSEHNKLKASQARDLLSRMLVIDPEKRISVDDALLHPYINVWYDESEVNAPAPGPWDHSVDEREHTVDQWKELIYQEVMEYDPTTAAAASANEPQPIR

SEQ ID NO: 2 - DNA, 1194 bp, Aphis gossypii
  CDS (1)..(1191)
atgctcggaacacaatcaaggtttgttcaaagacctcaaaataaaccagacactatgactgaattagctgatatgttctacactttagtatttggtgacactgaattttcagttcccagacgatatgttgatcttgtgcccaaaggtttaggtgctcagggcatggtggttgctgctatggacactattactcaacaaagcgttgcaattaaaaaactatctagaccctttcaaaatgtaacacatgcaaaaagagcatatagagagttcaaacttatgaaattagtaaatcataaaaatattattggtttactaaatgccttcacgccacaaaggacattagatgattttcaagatgtgtacttggtaatggaactgatggatgctaatctatgccaggtgatccaaatggatcttgatcatgaacgaatgtcatatcttttatatcaaatgttatgtggtattaaacatttacattcagcaggaattattcatagagaccttaaacctagtaatattgttgtaaaatctgactgcacattaaagattttggattttggattagctcgaacagctggaaccacatttatgatgacgccatatgttgttactagatactatcgtgctccagaggtaattttaggtatgggttataaggaaaatgttgacatttggtcagtaggttgtattatgggtgagatgataagaggtggcgttttatttcctggaactgatcatatagatcaatggaataaaattatagaacaactgggcacaccttcacaagaatttatgagaagattacaaccaactgttagaaattatgttgaaaatagacctcgttatccaggttattcatttgatcgattatttcctgacgtactatttccgtcagattcgtctgaacataataaacttaaagcaagccaagctcgtgaccttttgtctcgaatgctggttatcgatccagaaaagcgtatatctgtggacgatgcattgctacatccatatataaatgtttggtatgatgaaagtgaagtaaatgcaccagcaccaggaccatgggaccattcagtagatgaacgtgaacataccgttgatcaatggaaagaacttatctatcaagaagtcatggagtatgatccaactactgcagcagcagcttctgctaatgaaccacaaccaatccgatag

SEQ ID NO: 3 - DNA, 29 bp, Artificial Sequence
  Description of Artificial Sequence Synthetic primer
gtcagacattggacggaggtaaactgtgc

SEQ ID NO: 4 - DNA, 25 bp, Artificial Sequence
  Description of Artificial Sequence Synthetic primer
gtttgttgaatttgagattgtccac

SEQ ID NO: 5 - DNA, 34 bp, Artificial Sequence
  Description of Artificial Sequence Synthetic primer
ggatccatgctcggaacacaatcaaggtttgttc

SEQ ID NO: 6 - DNA, 33 bp, Artificial Sequence
  Description of Artificial Sequence Synthetic primer
cttataacccatacctaaaattacctctggagc

SEQ ID NO: 7 - DNA, 33 bp, Artificial Sequence
  Description of Artificial Sequence Synthetic primer
gctccagaggtaattttaggtatgggttataag

SEQ ID NO: 8 - DNA, 17 bp, Artificial Sequence
  Description of Artificial Sequence Synthetic primer
caggaaacagctatgac

SEQ ID NO: 9 - DNA, 17 bp, Artificial Sequence
  Description of Artificial Sequence Synthetic primer
gttttcccagtcacgac

SEQ ID NO: 10 - DNA, 38 bp, Artificial Sequence
  Description of Artificial Sequence Synthetic primer
cgctttgttgagtaatagtgtccatagcagcaaccacc

SEQ ID NO: 11 - DNA, 38 bp, Artificial Sequence
  Description of Artificial Sequence Synthetic primer
ggtggttgctgctatggacactattactcaacaaagcg

SEQ ID NO: 12 - DNA, 39 bp, Artificial Sequence
  Description of Artificial Sequence Synthetic primer
gtggttccagctgttcgagctaatccaaaatccaaaatc

SEQ ID NO: 13 - protein, 21 aa, Artificial Sequence
  Description of Artificial Sequence Synthetic peptide for JNK substrate
KRELVEPLTPSGEAPNQALLR

SEQ ID NO: 14 - protein, 22 aa, Artificial Sequence
  Description of Artificial Sequence Synthetic peptide for JNK substrate
KRELVEPLTPSGEAPNQALLRC

SEQ ID NO: 15 - DNA, 732 bp, Blattella germanica
  CDS (1)..(732)
  misc_feature (10)..(12): This codon may encompass “aaa” or “aca” and codes for Lys or Thr
atgttctacamagtggagtttggagacactgagttctctgttcccaatcgctattcggacttggcacccaaaggggtgggtgcgcagggcatggtctgtgctgcttatgacacagtgactcagcagaacgtggctatcaagaaattgagccgtccattccagaatgttacgcatgctaagcgygcctatcgagaattcaagctcatgaagctggttaatcacaagaatattattggcctgctaaatgctttcacaccacaaagatcattagaggagtttcaagatgtttatctagtaatggagcttatggatgccaacctctgccaggtcatccaaatggacctagatcatgagaggatgtcttatcttctctatcagatgcthtgcggcatcaagcatctccactctgcaggcatcatacatagrgacctgaaamctagcaacattgtggtaaaatcagactgtaccctgaaaatattagactttggtttggcaagaacggctggcacaacattcatgatgactccatatgttgttacgcgctactatagggcaccagaggtaatattaggtatgggatacaaagaaaatgtggacatctggtcagtagggtgcatcatgggtgagatgatacgaggtggagtgttgtttccagggacagatcacattgatcagtggaataaaataatagaacaacttggtactccatcacaagactttaggaaacgactc

SEQ ID NO: 16 - protein, 244 aa, Blattella germanica
  MOD_RES (4)..(4): Lys or Thr
MFYXVEFGDTEFSVPNRYSDLAPKGVGAQGMVCAAYDTVTQQNVAIKKLSRPFQNVTHAKRAYREFKLMKLVNHKNIIGLLNAFTPQRSLEEFQDVYLVMELMDANLCQVIQMDLDHERMSYLLYQMLCGIKHLHSAGIIHRDLKXSNIVVKSDCTLKILDFGLARTAGTTFMMTPYVVTRYYRAPEVILGMGYKENVDIWSVGCIMGEMIRGGVLFPGTDHIDQWNKIIEQLGTPSQDFRKRL

SEQ ID NO: 17 - protein, 6 aa, Artificial Sequence
  Description of Artificial Sequence Synthetic 6xHis tag
HHHHHH

(19) United States
(12) Patent Application Publication
(10) Pub. No.: US 2011/0053883 A1
(43) Pub. Date: Mar. 3, 2011

(54) AGENT THAT MODULATES PHYSIOLOGICAL CONDITION OF PESTS, INVOLVED IN INSECT C-JUN AMINO-TERMINAL KINASE ACTIVITY

(75) Inventors: Yasutaka Shimokawatoko, Chiba (JP); Mar Van De Craen, Aalter (BE); Irene Nooren, Utrecht (NL); Sandra Turconi, Hertforshire (GB); Annelies Roobrouck, Mater (BE); Wendy Maddelein, Gijzenzele (BE)

(73) Assignee: SUMITOMO CHEMICAL COMPANY, LIMITED, CHOU-KU, TOKYO (JP)

(21) Appl. No.: 11/993,602

(22) PCT Filed: Jun. 23, 2006
(86) PCT No.: PCT/JP2006/313042

§ 371 Date: Aug. 31, 2009

(30) Foreign Application Priority Data
Jun. 23, 2005 (JP) 2005-183032

(51) Int. Cl. A01N 43/90; C12Q 1/48; C12P 19/34; C12N 9/12; C12N 7/01; C12N 5/10; C12N 15/63; A01N 57/36; A01N 43/56; A01N 43/38; A01N 43/30; A01N 41/04; C07H 21/04; C07H 19/19; C07D 487/04; C07D 231/54; C07D 403/14; C07D 493/10; C07C 309/67; C07F 9/6584; C07H 21/00; A01P 7/04

(52) U.S. Cl. 514/45; 435/15; 435/91.2; 435/455; 435/194; 435/235.1; 435/325; 435/348; 435/320.1; 514/110; 514/263.4; 514/406; 514/422; 514/462; 514/517; 536/23.2; 536/24.33; 536/27.4; 544/262; 548/356.5; 548/455; 549/334; 558/55; 564/13

(57) ABSTRACT

The present invention provides an agent that modulates physiological condition of pests, wherein the agent has an ability to modulate the activity of an insect c-Jun NH₂-terminal kinase; a method for assaying pesticidal activity of a test substance, which comprises a step of measuring the activity of a c-Jun NH₂-terminal kinase in a reaction system in which the c-Jun NH₂-terminal kinase contacts with a test substance, and the like.

### TECHNICAL FIELD

The present invention relates to an agent that modulates physiological condition of pests, involved in insect c-Jun NH₂-terminal kinase activity, and the like.

### BACKGROUND ART

Regarding insect c-Jun NH₂-terminal kinase, for example, it has been suggested:

that the D. melanogaster c-Jun NH₂-terminal kinase, encoded by the bsk gene, is involved in different physiological processes as early as the embryonic stage, and bsk mediates an immune response in cells in culture and morphogenesis in viva (for example, Sluss et al., Genes Dev. 10 (21): 2745-58, 1996);

that the developmental signaling protein wingless (wg) and c-Jun NH₂-terminal kinase signaling cascades collaborate to promote both dorsal closure and ventral patterning (for example, McEven at al., Development; 127 (16): 3607-17, 2000);

that c-Jun NH₂-terminal kinase signaling cascades are not necessary for normal wing development, but only when a level of the signal is not normal, the cascades are necessary as information for development of wing at a normal position, this is not seen at development where c-Jun NH₂-terminal kinase signaling cascades are normal, but when the signaling cascades become abnormal, the kinase is activated for providing a system for maintaining suitable development (for example, Adachi-Yamada et al., Nature, 400 (6740): 166-9, 1999), and the like.

In addition, in D. melanogaster a null mutant phenotype is described as embryonic recessive lethal, which is in accordance with central function of insect c-Jun NH2-terminal kinase. Mutations have been isolated which affect the embryonic dorsal cuticle and the embryonic dorsal epidermis.

During D. melanogaster development, the c-Jun NH₂-terminal kinase signal transduction pathway regulates morphogenetic tissue closure movements that involve cell shape changes and reorganization of the actin cyto-skeleton (for example, Noselli et al. Curr Opin Genet Dev., 9 (4): 466-72, 1999). Also in D. melanogaster, c-Jun NH₂-terminal kinase loss-of-function mutations have been described which are lethal at the embryonic stage. (for example, Sluss et al., Genes Dev. 10 (21): 2745-58, 1996).

Discovery of agricultural chemicals has traditionally been based on a random screening process, often directly testing the effects of specific chemicals on whole organisms, such as insects, fungi and/or plants and determining biological activity. Once chemical compounds with the appropriate biological activity are discovered, more intense research is required to specifically determine the mode of action or site of action of these compounds at the molecular level, in order to predict safety and environmental load of these compounds.

### DISCLOSURE OF INVENTION

This invention describes a more target-based approach of screening agricultural chemicals, whereby compounds are screened against a specific target that has been identified as biologically and/or physiologically relevant with intent of chemically interfering with the target site to control insects or other, pest organisms.

Specifically, this invention describes that an agent that modulates physiological condition of pests and having an ability to modulate the activity of an insect c-Jun NH₂-terminal kinase is useful to control pests.

That is, the present invention provides:

1. An agent that modulates physiological condition of pests, wherein said agent has an ability to modulate the activity of an insect c-Jun NH₂-terminal kinase;

2. An agent according to item 1, wherein said c-Jun NH₂-terminal kinase is a cotton aphid c-Jun NH₂-terminal kinase;

3. An agent according to item 1, wherein said agent is a pesticidal agent;

4. An agent according to item 1, wherein said ability to modulate the activity of an insect c-Jun NH₂-terminal kinase is an ability to inhibit a reaction of the insect c-Jun NH₂-terminal kinase with the peptide of SEQ ID NO: 14:

5. A pesticidal agent which comprises a substance that has an ability to modulate the activity of an insect c-Jun NH₂-terminal kinase or an agriculturally acceptable salt of the substance as an active ingredient:

6. A pesticidal agent according to item 5, wherein said substance has an ability to inhibit a reaction of the insect c-Jun NH₂-terminal kinase with the peptide of SEQ ID NO: 14;

7. A pesticidal agent according to item 6, wherein said substance has an ability to inhibit the reaction of the insect c-Jun NH₂-terminal kinase with the peptide of SEQ ID NO: 14 in a cell-free system, wherein in the presence of said substance of 10 micro M or more the activity of said c-Jun NH₂-terminal kinase is lower than that in the absence of said substance;

8. A pesticidal agent according to item 6, wherein said substance has an ability to inhibit a reaction of the insect c-Jun NH₂-terminal kinase with the peptide of SEQ ID NO: 14 in a cell-free system with an IC₅₀ of 100 micro VI or less;

9. A method for assaying pesticidal activity of a test substance, which comprises;

(1) a first step of measuring the activity of a c-Jun NH₂-terminal kinase selected from the following group. A in a reaction system in which said c-Jun NH₂-terminal kinase contacts with a test substance; and

(2) a second step of evaluating the pesticidal activity of said test substance based on the difference obtained by comparing the activity measured in the first step with the activity of a control:

### <Group A>

(a) a protein comprising the amino acid sequence of SEQ ID NO: 1;

(b) a protein comprising an amino acid sequence with deletion, addition or substitution of one or more amino acids in the amino acid sequence of SEQ ID NO: 1, wherein said protein has c-Jun NH₂-terminal kinase activity;

(c) a protein comprising an amino acid sequence that has sequence identity of 83% or more to the amino acid sequence of SEQ ID NO: 1, wherein said protein has c-Jun NH₂-terminal kinase activity;

(d) a protein comprising the amino acid sequence encoded by the nucleotide sequence of SEQ ID NO: 2;

(e) a protein comprising an amino acid sequence encoded by a nucleotide sequence that has sequence identity of 65% or more to the nucleotide sequence of SEQ ID NO: 2, wherein said protein has c-Jun NH₂-terminal kinase activity;

(f) a protein comprising an amino acid sequence encoded by a polynucleotide, wherein said polynucleotide hybridizes under a stringent condition to a polynucleotide comprising a nucleotide sequence complementary to the nucleotide sequence of SEQ ID NO: 2, and wherein said protein has c-Jun NH₂-terminal kinase activity;

(g) a protein comprising an amino acid sequence of an insect c-Jun NH₂-terminal kinase; and

(h) a protein comprising an amino acid sequence of a cotton aphid c-Jun NH₂-terminal kinase;

10. A method for screening a pesticidal substance, which comprises selecting a substance having the pesticidal activity that is evaluated by the method according to item 9;

11. A pesticidal agent which comprises a substance selected by the method according to item 10 or agriculturally acceptable salts thereof as an active ingredient;

12. A method for controlling pests which comprises applying an effective amount of the pesticidal agent according to item 5, 6, 7, 8 or 11 to the pest, habitat of the pest or plant to be protected from the pest;

13. A method for controlling pests which comprises:

identifying a substance having the pesticidal activity that is evaluated by the method according to item 9, and

contacting the pest with the identified pesticidal substance;

14. An insect c-Jun NH₂-terminal kinase comprising an amino acid sequence selected from the following group B:

### <Group B>

(a) the amino acid sequence of SEQ ID NO: 1:

(b) an amino acid sequence with deletion, addition or substitution of one or more amino acids in the amino acid sequence of SEQ ID NO: 1, wherein said amino acid sequence has c-Jun NH₂-terminal kinase activity;

(c) an amino acid sequence that has sequence identity of 95% or more to the amino acid sequence of SEQ ID NO: 1, wherein said amino acid sequence has c-Jun NH₂-terminal kinase activity;

(d) the amino acid sequence encoded by the nucleotide sequence of SEQ ID NO: 2;

(e) an amino acid sequence encoded by a nucleotide sequence that has sequence identity of 75% or more to the nucleotide sequence of SEQ ID No: 2, wherein said amino acid sequence has c-Jun NH₂-terminal kinase activity;

(f) an amino acid sequence encoded by a polynucleotide, wherein said polynucleotide hybridizes under a stringent condition to a polynucleotide comprising a nucleotide sequence complementary to the nucleotide sequence of SEQ ID NO: 2, wherein said amino acid sequence has c-Jun NH₂-terminal kinase activity; and

(g) an amino acid sequence of a cotton aphid c-Jun NH₂-terminal kinase;

15. Use of an insect c-Jun NH₂-terminal kinase as a reagent that provides an indicator to evaluate pesticidal activity;

16. Use of an insect c-Jun NH₂-terminal kinase according to item 14 as a reagent that provides an indicator to evaluate pesticidal activity;

17. A polynucleotide which comprises a nucleotide sequence encoding an amino acid sequence of a c-Jun NH₂-terminal kinase according to item 14;

18. A polynucleotide according to item 17, which comprises the nucleotide sequence of SEQ ID NO 2:

19. A polynucleotide which comprises a nucleotide sequence complementary to a nucleotide sequence of a polynucleotide according to item 17 or 18;

20. A polynucleotide which comprises:

a partial nucleotide sequence of a polynucleotide according to item 17 or 18; or

a nucleotide sequence complementary to said partial nucleotide sequence;

21. A polynucleotide according to item 20, which comprises a nucleotide sequence of SEQ ID NO: 3 or 4:

22. A method for obtaining a polynucleotide comprising a nucleotide sequence encoding an amino acid sequence of a c-Jun NH₂-terminal kinase, which comprises:

a step of amplifying a desired polynucleotide by polymerase chain reaction using as a primer a polynucleotide according to item 20 or 21;

a step of identifying the desired polynucleotide amplified; and

a step of recovering the identified polynucleotide;

23. A method for obtaining a polynucleotide comprising a nucleotide sequence encoding an amino acid sequence of a c-Jun NH₂-terminal kinase, which comprises:

a step of detecting a desired polynucleotide by hybridization using as a probe a polynucleotide according to item 19, 20 or 21;

a step of identifying the desired polynucleotide detected; and

a step of recovering the identified polynucleotide;

24. A circular polynucleotide comprising a nucleotide sequence of a polynucleotide according to item 17 or 18, wherein said nucleotide sequence is operably linked to a baculovirus promoter;

25. A circular polynucleotide according to item 24, wherein said promoter is a polyhedrin gene promoter;

26. A circular polynucleotide according to item 24 or 25, wherein said polynucleotide comprises a replication origin for autonomous replication in a host cell;

27. A circular polynucleotide according to item 24, 25 or 26, wherein said polynucleotide comprises a nucleotide sequence of a baculovirus shuttle vector and capable of propagating as a virus in an insect cell;

28. A method for producing a circular polynucleotide, which comprises ligating a polynucleotide according to item 17 or 18 into a vector;

29. A transformant in which a polynucleotide according to item 17 or 18 is introduced;

30. A transformant according to item 29, wherein said transformant is a transformed insect cell;

31. A method for producing a transformant, which comprises introducing a polynucleotide according to item 17 or 18 into a host cell;

32. A recombinant baculovirus comprising within its genome a polynucleotide according to item 17 or 18;

33. A method for producing a c-Jun NH₂-terminal kinase, which comprises a step of culturing the transformant according to item 29 or 30 and recovering a produced c-Jun NH₂-terminal kinase;

34. Use of a c-Jun NH₂-terminal kinase according to item 14 or a polynucleotide according to any one of items 17 to 21 as a research tool;

35. Use according to item 34, wherein the research tool is an experimental tool for screening a pesticidal substance; and

36. A system which comprises;

a means to input, store and manage a data information of an ability of test substances, wherein said ability is an ability to modulate the activity of an insect c-Jun NH₂-terminal kinase;

a means to query and retrieve the data information based on a desired criterion; and

a means to display and output the result which is queried and retrieved.

### MODES FOR CARRYING OUT THE INVENTION

The present invention will be explained in detail below.

In the present invention, the “pests” indicates small animals which cause harm or discomfort to life of the people by harming man and animals directly or by damaging crops. Examples thereof include arthropod such as insects, mites and ticks and Nematoda, and typical examples of which are as follows:

### Hemiptera:

Delphacidae such as Laodelphax striatellus, Nilaparvata lugens and Sogatella furcifera, Deltocephalidae such as Nephotettix cincticeps and Empoasca onukii. Aphididae such as Aphis gossypii and Myzus persicae, Pentatomidae, Aleyrodidae such as Trialeurodes vaporariorum, Bemisia tabaci and Bemisia argentifolli, Coccidae, Tingidae, Psyllidae, etc.

### Lepidoptera:

Pyralidae such as Chilo suppressalis, Cnaphalocrocis medinalis, Ostrinia nubilalis and Parapediasia teterrella, Noctuidae such as Spodoptera litura, Spodoptera exigua, Pseudaletia separata, Mamestra brassicae, Agrotis ipsilon, Trichoplusia spp., Heliothis spp., Helicoverpa spp. and Earias spp., Pleridae such as Pieris rapae crucivora, Tortricidae such as Adoxophyes orana fasciata, Grapholita molesta and Cydia pomonella, Carposinidae such as Carposina niponensis, Bucculatricidae such as Lyonetia clerkella, Gracillariidae such as Phyllonorycter ringoniella. Phyllocnistidae such as Phyllocnistis citrella, Yponomeutidae such as Plutella xylostella, Gelechiidae such as Pectinophora gossypiella, Arctiidae, Tineidae, etc.

### Diptera:

Culex such as Culex pipiens pallens, Culex tritaeniorhynchus and Culex quinquefasciatus, Aedes such as Aedes aegypti and Aedes albopictus, Anopheles such as Anophelinae sinensis, Chironomidae. Muscidae such as Musca domestics and Muscina stabulans, Calliphoridae, Sarcophagidae, Fannia canicularis, Anthomyiidae such as Delia Platura and Delia antigua, Trypetidae, Drosophilidae, Psychodidae, Simuliidae, Tabanidae, Stomoxyidae, Agromyzidae, etc.

### Coleoptera:

Diabrotica such as Diabrotica virgifera virgifera and Diabrotica undecimpunctata howardi, Scarabaeidae such as Anomala cuprea and Anomala rufocuprea, Curculionidae such as Sitophilus zeamais, Lissorphoptrus oryzophilus and Calosobruchys chinensis, Tenebrionidae such as Tenebrio molitor and Tribolium castaneum, Chrysomelidae such as Oulema oryzae, Aulacophora femoralis, Phyllotreta striolata and Leptinotarsa decemlineata, Anobiidae, Epilachna spp. such as Epilachna vigintioctopunctata, Lyctidae, Bostrychidae, Cerambycidae, Paederus fuiscipes, etc.

### Thysanoptera:

Thripidae such as Thrips spp. including Thrips palmi, Frankliniella spp. including Frankliniella occidentalis and Sciltothrips spp. including Sciltothrips dorsalis, Phlaeothripidae, etc.

### Hymenoptera:

### Tenthredinidae, Formicidae, Vespidae, etc.

### Dictyoptera:

### Blattidae, Blattellidae, etc.

### Orthoptera:

### Acrididae, Grylletalpidae etc.

### Siphonaptera:

Pulex irritans, etc.

### Anoplura:

Pediculus humanus capitis, etc.

### Isoptera:

### Termitidae, etc.

### Acarina:

Tetranychidae such as Tetranychus urticae, Tetranychus kanzawai, Panonychus citri, Panonychus ulmi, and Oligonychus spp., Eriophyidae such as Aculops pelekassi and Aculus schlechtendali. Tarsonemidae such as Polyphagotarsonemus latus, Tenuipalpidae, Tuckerellidae, Ixodidae such as Haemaphysalis longicornis, Haemaphysalis flava, Dermacentor taiwanicus, Ixodes ovatus, Ixodes persulcatus and Boophilus microplus, Acaridae such as Tyrophagus putrescentiae, Dermanyssidae, Cheyletidae such as Dermatophagoides farinae and Dermatophagoides ptrenyssnus, such as Cheyletus eruditus, Cheyletus malaccensis and Cheyletus moorei, Dermanyssus spp., etc.

### Nematodes:

Pratylenchus coffeae, Pratylenchus fallax, Heterodera glycines, Globodera rostochiensis, Meloidogyne hapla, Meloidogyne incognita, etc.

In the present invention, the “modulate physiological condition of pests” indicates changing condition such as various phenomena in a living body which are maintained for living in pests, for example, function such as aspiration, digestion, secretion, body liquid circulation, metabolism, neurotransmission and the like, or mechanism thereof into condition apart from usual condition. Examples include changing condition by cessation of aspiration so that oxygen necessary for internal metabolism of pests is not supplied, and changing condition by cessation of function of neurotransmission of pests so that various movements of pests are ceased.

In the present invention, the “agent which modulates physiological condition of pests” is an agent which can modulate physiological condition of pests when being applied to pests.

In the present invention, the “insect c-Jun NH₂-terminal kinase” indicates a c-Jun NH₂-terminal kinase that occurs in insect, among c-Jun NH₂-terminal kinase present in various organisms. Herein, insect is an animal classified under Animalia, Arthropoda. Insecta, and examples of which include arthropod of the order Protura, Collembola, Diplura, Thysanura, Ephemeroptera, Odonata, Plecoptera, Grylloblattodea, Orthoptera, Phasmatodea, Dermaptera, Mantodea, Elattaria, Isoptera, Embioptera, Psocoptera, Mallophaga, Anoplura, Thysanoptera, Hemiptera, Neuroptera, Mecoptera, Trichoptera, Lepidoptera, Coleoptera, Diptera, Hymenoptera, Siphonaptera, Strepsiptera, and the like.

C-Jun amino-terminal kinase (EC 2.7.1.37) is a protein kinase, and is a member of MAP kinase family having activity of transferring a phosphate group of ATP to c-Jun.

Several assays well known to a person skilled in the art may be used to monitor c-Tun NH₂-terminal kinase activity. These assays use natural substrates or synthetic peptides-substrates. For example, the c-Jun NH₂-terminal kinase activity can be monitored using a radioactivity based assay with biotinylated c-Jun Fusion protein as substrate. Another example to monitor the c-Jun NH₂-terminal kinase activity using c-Jun Fusion Protein is the Kinase-Glo Luminescent Kinase Assay. A third example to monitor the c-Jun NH₂-terminal kinase activity using a synthetic peptide substrate is fluorescence polarization. ATF-2 (activating transcription factor 2) is an example of a substrate of c-Jun NH₂-terminal kinase (Fowler Ann et al., (2002), Analytical Biochemistry 308 (2002) 223-231).

It is thought that 9-12 residues in the peptide make contact with the active site cleft in Ser/Thr protein kinases, although peptides as small as 7-mars have been shown to bind. The specificity of peptide substrates is generally restricted to residues +3 to −3 of the phosphorylation site, although residues outside of this region can occasionally contribute to substrate specificity. A peptide substrate of the kinase can be selected from sequence and literature information of known endogenous substrates. Using the method, a plurality of different peptide substrates are designed. For example, activity on the substrate of a cotton aphid-derived c-Jun amino-terminal kinase is measured by a radioactivity-based method. From those measurement results, for example, a substrate having highest phosphate group transference activity may be selected. As such the substrate, for example, there is a peptide consisting of the amino acid sequence of SEQ ID NO: 14.

A selected peptide substrate can be used in a radioactivity-based method as described above and, for example, activity of a c-Jun NH₂-terminal kinase can be measured using a fluorescent polarization method using the IMAP technology. The IMAP technology is based on the high affinity binding of phosphate by immobilized metal (M^{III}) coordination complexes on nanoparticles. This IMAP binding reagent complexes with phosphate groups on fluorescent phosphopeptides generated in a c-Jun NH₂-terminal kinase reaction. Such binding causes a change in the rate of the molecular motion of the peptide, and results in an increase in the fluorescence polarization value observed for the fluorescent label attached at the end of the peptide.

Among the aforementioned various methods of measuring activity of a c-Jun NH₂-terminal kinase, as a method for automatic and effective measurement of the c-Jun NH₂-terminal kinase activity of many samples, the aforementioned fluorescent polarization method using the IMAP technology using, as a substrate, a peptide consisting of the amino acid sequence of SEQ ID NO: 14 is desirable. Examples include a method using IMAP screening Express Kit with progressive Binding system (manufactured by Molecular Device) according to a method described in the manual annexed to the system and employing, as a substrate, a peptide comprising the amino acid sequence of SEQ ID NO: 13 to which biotin was added at the amino-terminus, and a peptide comprising the amino acid sequence of SEQ ID NO: 14 in which the carboxyl-terminus was fluorescently labeled.

In addition, a method of measuring activity of an insect c-Jun NH₂-terminal kinase can be performed by a similar method to that described above.

Several amino acid sequences of c-Jun NH₂-terminal kinase have been identified in different insect species, for example in D. melanogaster (accession No. AAB51187), in Anopheles gambiae (accession No. EAA05905), in Aedes albopictus (accession No. AA031950), in Apis mellifera (accession No. XP_{—}392806), and the like, which can be found in public databases. Also, several nucleotide sequences of c-Jun NH₂-terminal kinase genes have been identified in different insect species, for example in D. melanogaster (accession No. NM_{—}164900), in Anopheles gambiae (accession No. XM_{—}310236), in Aedes albopictus (accession No. AF515780), in Apis mellifera (accession No. XM_{—}392806), and the like, which can be found in public databases.

In addition, according to the methods described below, an amino acid sequence of c-Jun NH₂-terminal kinase and a nucleotide sequence of c-Jun NH₂-terminal kinase gene can be identified from a cotton aphid. The identified amino acid sequence of cotton aphid c-Jun NH₂-terminal kinase is shown in SEQ ID NO: 1, and the nucleotide sequence of cotton aphid c-Jun NH₂-terminal kinase gene is shown in SEQ ID NO: 2.

Several amino acid sequences of c-Jun NH₂-terminal kinase have been identified in animals other than insect, for example in Caenorhabditis elegans (accession No. NP_{—}741434), in Homo sapiens (accession No. NP_{—}002744, NF_{—}620448, NP_{—}620446), and the like, which can be found in public databases. Also, several nucleotide sequences of c-Jun NH₂-terminal kinase genes have been identified in animals other than insect, for example in Caenorhabditis elegans (accession No. NM_{—}171371), in Homo sapiens (accession No. NM 002753, NM 13$982, NM_{—}138980), and the like, which can be found in public databases.

Table 1 shows Sequence identity of the amino acid sequence of cotton aphid c-Jun NH₂-terminal kinase (SEQ ID NO: 1) and the nucleotide sequence of cotton aphid c-Jun NH₂-terminal kinase gene (SEQ ID NO: 2) with the sequence of c-Jun NH₂-terminal kinase and gene thereof found in other animals.

TABLE 1

| | Identity of | Identity of |
|---|---|---|
| | amino acid | nucleotide |
| | sequence (%) vs | sequence (%) vs |
| Origin of sequence | SEQ ID NO: 1 | SEQ ID NO: 2 |
| Drosophila melanogaster | 84.0 | 71.3 |
| Apis mellifera | 88.4 | 69.6 |
| Aedes albopictus | 92.1 | 69.4 |
| Anopheles gambiae | 88.5 | 67.9 |
| Ceanorhabditis elegans | 71.8 | 60.6 |
| Homo sapiens JNK1 | 82.4 | 61.8 |
| Homo sapiens JNK2 | 80.0 | 62.9 |
| Homo sapiens JNK3 | 62.2 | 58.8 |

An ability to modulate the activity of an insect c-Jun NH₂-terminal kinase refers to an ability to increase or decrease activity of an insect c-Jun NH₂-terminal kinase, that is, means an ability to activate a c-Jun NH₂-terminal kinase, or an ability to inhibit activity of a c-Jun NH₂-terminal kinase. And, a test substance can be added to the reaction system for measuring c-Jun NH₂-terminal kinase activity to investigate influence of the test Substance on the c-Jun NH₂-terminal kinase activity.

As a substance having an ability to inhibit the activity of a c-Jun NH₂-terminal kinase, SP600125 (Bennett B L et al., Proc Natl Acad Sci USA. 2001 Nov. 20; 98(24):13681-6.), and the like are known.

An IC₅₀ value of a test substance in the reaction means a concentration of a test substance at which 50% of the activity of the reaction with no test substance is inhibited. The IC₅₀ value of a test substance can be determined by adding test substances of different concentrations to the c-Jun NH₂-terminal kinase activity measuring reaction system, measuring the c-Jun NH₂-terminal kinase activity (response) at each concentration of added test substance (dose), producing a dose-response curve, and Calculating a concentration of the added test substance, at which the c-Jun NH₂-terminal kinase activity is 50% inhibited. More specifically, a dose-response curve may be produced using 4 Parameter Logistic Model or Sigmoidal Dose-Response Model:

$$f ( x ) = ( A + ( ( B - A ) / ( 1 + ( ( C / x ) {}^{-} D ) ) ) \\ f ( x ) = A + \frac{B - A}{1 + {( C / x )}^{D}}$$

to calculate the IC₅₀. Practically, the IC₅₀ value may be calculated using XLfit (manufactured by IDBS) which is a commercially available calculating software.

An agent that has an ability to modulate the activity of an insect c-Jun NH₂-terminal kinase is an agent containing as an active ingredient a substance having an ability to modulate the activity of an insect c-Jun NH₂-terminal kinase.

In the present invention, the “agent that modulates physiological condition of pests, wherein the agent has an ability to modulate the activity of an insect c-Jun NH₂-terminal kinase” is an agent having an ability to modulate the activity of insect c-Jun NH₂-terminal kinase specified by the aforementioned measuring method, and means an agent that can modulate physiological condition of pests. Preferable examples of the agent include an agent in which an insect c-Jun NH₂-terminal kinase is a cotton aphid c-Jun NH₂-terminal kinase. In addition, preferable examples of the agent include an agent in which an agent that modulates physiological condition of pests is a pesticidal agent. In addition, preferable examples of the agent include an agent in which an ability to modulate the activity of an insect c-Jun NH₂-terminal kinase is en ability to inhibit a reaction using the peptide of SEQ ID NO. 14 as a substrate.

In the present invention, the “pesticidal agent” indicates an agent having an ability to control the pests. Examples of a method for measuring an ability to control pests include, in addition to the methods disclosed in the present invention, a method of measuring pesticidal activity on the pests. Specifically, for example, the pesticidal activity can be measured according to the following method.

According to the method described in Handbook of Insect Rearing Vol 0.1 (Elsevier Science Publishers 1985), pp. 35 to pp. 36 except that a sterilized artificial feed having the following composition (Table 2) is prepared, and a solution of a test agent in DMSO is added at 0.5% by volume of the artificial feed and is mixed, a cotton aphid is reared, the number of surviving cotton aphids is investigated after 6 days, and a controlling value is obtained according to the following equation.

TABLE 2

| | (mg/100 ml) |
|---|---|
| Amino acid | |
| L-Alanine | 100.0 |
| L-arginine | 275.0 |
| L-Asparagine | 550.0 |
| L-Aspartic acid | 140.0 |
| L-cysteine | 40.0 |
| (hydrochloride) | |
| L-glutamic acid | 140.0 |
| L-glutamine | 150.0 |
| L-glycine | 80.0 |
| L-histidine | 80.0 |
| L-isoleucine | 80.0 |
| L-leucine | 80.0 |
| L-lysine | 120.0 |
| (hydrochloride) | |
| L-methionine | 80.0 |
| L-phenylalanine | 40.0 |
| chloride | |
| L-proline | 80.0 |
| chloride | |
| L-serine | 80.0 |
| L-threonine | 140.0 |
| L-tryptophan | 10 80.0 |
| L-tyrosine | 40.0 |
| L-valine | 80.0 |
| Vitamins | |
| Ascorbic acid | 100.0 |
| Biotin | 0.1 |
| Calcium | 5.0 |
| pantothenate | |
| Choline | 50.0 |
| chloride | |
| Inositol | 50.0 |
| Nicotinic acid | 10.0 |
| Thiamine | 2.5 |
| Others | |
| Sucrose | 12500.0 |
| Dipotassium | 1500.0 |
| hydrogen | |
| phosphate | |
| Magnesium | 123.0 |
| sulfate | |
| Cupric | 0.2 |
| chloride | |
| Ferric | 11.0 |
| chloride | |
| Manganese | 0.4 |
| Zinc sulfate | 0.8 |
| (anhydrous) | |

Adjusted to pH 6.8
Controlling value (%) = {1 − (Cb × Tai)/(Cai × Tb)} × 100
Letters in the equation represent the following meanings.
Cb: Number of surviving worms before treatment in non-treating section
Cai: Number of surviving worms at observation in non-treated section
Tb: Number of surviving worms before treatment in non-treated section
Tai: Number of surviving worms at observation in a treated section

It may be said that a test agent exhibiting a significantly high controlling value has the pesticidal activity. More preferably, it may be determined that a test agent having the controlling value of 30% or more has substantial pesticidal activity, and it may be determined that a test agent having the controlling value of less than 30% has no substantial pesticidal activity.

The pesticidal agent in the present invention contains a chemical substance having an ability to modulate the activity of insect c-Jun NH₂-terminal kinase or an agriculturally acceptable salt thereof as an active ingredient.

In the present invention, an agriculturally acceptable salt refers to a salt in such a form that preparation of a controlling agent and application of the preparation do not become impossible, and may be a salt in any form. Specifically, examples of the salt include acid addition salts with mineral acids such as hydrochloric acid, hydrobromic acid, hydriodic acid, sulfuric acid, nitric acid, and phosphoric, organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethansulfonic acid, or acidic amino acids such as aspartic acid and glutamic acid; salts with inorganic bases such as sodium, potassium, magnesium, and aluminum, organic bases such as methylamine, ethylamine, and ethanolamine, or basic amino acids with lysine and ornithine; and an ammonium salts.

In the present invention, the “pesticidal agent which comprises a substance having an ability to modulate the activity of an insect c-Jun NH₂-terminal kinase or a an agriculturally acceptable salt thereof as an active ingredient” means an agent which can control pests by containing a substance having an ability to modulate the activity of insect c-Jun NH₂-terminal kinase identified in the measuring method or an agriculturally acceptable salt thereof as an active ingredient. Preferable examples of the substance include a compound having an ability to inhibit a reaction) of a c-Jun NH₂-terminal kinase with the peptide of SEQ ID NO: 14. More preferable examples of the substance include a substance having an ability to inhibit the reaction of the insect c-Jun NH₂-terminal kinase with the peptide of SEQ ID NO: 14 in a cell-free system, wherein in the presence of the substance of 10 μM or more the activity of the c-Jun NH₂-terminal kinase is lower than that in the absence of the substance. In addition, further preferable examples of the substance include a substance having an ability to inhibit a reaction of the insect c-Jun NH₂-terminal kinase with the peptide of SEQ ID NO: 14 in a cell-free system with an IC50 of 100 μM or less.

In the present invention, the “method for assaying pesticidal activity of a test substance, which comprises a first step of measuring the activity of a c-Jun NH₂-terminal kinase selected from the group A in a reaction system in which the c-Jun NH₂-terminal kinase contacts with a test substance, and a second step of evaluating the pesticidal activity of the test substance based on the difference obtained by comparing the activity measured in the first step with the activity of a control” indicates a method characterized by comprising the first step and the second step in various methods for assaying a pesticidal ability of a test substance.

Herein, the group A indicates:

(a) a protein comprising the amino acid sequence of SEQ ID NO: 1;

(b) a protein comprising an amino acid sequence with deletion, addition or substitution of one or more amino acids in the amino acid sequence of SEQ ID NO: 1, wherein said protein has c-Jun NH₂-terminal kinase activity;

(c) a protein comprising an amino acid sequence that has sequence identity of 83% or more to the amino acid sequence of SEQ ID NO: 1, wherein said protein has c-Jun NH₂-terminal kinase activity;

(d) a protein comprising the amino acid sequence encoded by the nucleotide sequence of SEQ ID NO: 2;

(e) a protein comprising an amino acid sequence encoded by a nucleotide sequence that has sequence identity of 65% or more to the nucleotide sequence of SEQ ID NO: 2, wherein said protein has c-Jun NH₂-terminal kinase activity:

(f) a protein comprising an amino acid sequence encoded by a polynucleotide, wherein said polynucleotide hybridizes under a stringent condition to a polynucleotide comprising a nucleotide sequence complementary to the nucleotide sequence of SEQ ID NO: 2, and wherein said protein has c-Jun NH₂-terminal kinase activity;

(g) a protein comprising en amino acid sequence of an insect c-Jun NH₂-terminal kinase; and

(h) a protein comprising an amino acid sequence of a cotton aphid c-Jun NH₂-terminal kinase.

The first step is a step of measuring the activity of a c-Jun NH₂-terminal kinase in the state where a c-Jun NH₂-terminal kinase is contacted with a test substance by adding the test substance to the aforementioned various c-Jun NH₂-terminal kinase activity measuring reaction systems. In addition, the second step is a step of comparing the activity at measurement of a test substance with the substance of a control, and evaluating a pesticidal ability based on the difference. Herein, a control means, for example, in the case where a test substance dissolved in a solvent is added to the reaction system, a test section in which only a solvent same as that used to dissolve the test substance is added.

A c-Jun NH₂-terminal kinase used in a method for assaying a pesticidal ability possessed by a test substance, having the first step and the second step, is a protein shown in the group A. Among proteins of the group A, a difference which can be recognized between an amino acid sequence of protein represented by (a) and amino acid sequences of proteins represented by (b), (c), (e) (f), (g) and (h) is deletion, substitution, addition or the like of a part of amino acids. These include, for example, deletion due to processing which the protein having an amino acid sequence represented by (a) undergoes in a cell. In addition, examples include deletion, substitution, addition and the like of an amino acid generated by naturally occurring gene mutation due to a spices difference or an individual difference of an organism from which the protein is derived, or gene mutation which is artificially introduced by a site-directed mutagenesis, a random mutagenesis, mutation treatment or the like.

The number of amino acids undergoing the deletion, substitution, addition or the like may be the number in a range that the peptidase activity of a c-Jun NH₂-terminal kinase can be found out. In addition, examples of substitution of an amino acid include substitution with an amino acid which is similar in characteristic in hydrophobicity, charge, pH and steric structure. Specific examples of the substitution include substitution in an group of (1) glycine, alanine; (2) valine, isoleucine, leucine: (3) aspartic acid, glutamic acid, asparagine, glutamine, (4) serine, threonine; (5) lysine, arginine; (6) phenylalanine, tyrosine and the like.

Examples of a procedure of artificially introducing the deletion, addition or substitution of an amino acid (hereinafter, collectively referred to as alteration of amino acid in some cases) include a procedure of introducing site-directed mutation into a DNA encoding an amino acid sequence represented by (a) and, thereafter, expressing this DNA by a conventional method. Herein, examples of a site-directed mutagenesis include a method utilizing amber mutation (gapped duplex method, Nucleic Acids Res., 12, 9441-9456 (1984)), a method by PCR using primers for mutation introduction, and the like. In addition, examples of a procedure of artificially altering an amino acid include a procedure of randomly introducing mutation into a DNA encoding an amino acid sequence represented by (a) and, thereafter, expressing this DNA by a conventional method. Herein, examples of a method of randomly introducing mutation include a method of performing PCR using a DNA encoding any of the aforementioned amino acid sequences as a template, and using a primer pair which can amplify each full length DNA at reaction condition under which an addition amount of each of dATP, dTTP, dGTP and dCTP used as a substrate is changed from a conventional concentration, or at reaction condition under which a concentration of Mg²⁺ promoting a polymerase reaction is increased from a conventional concentration. Examples of the procedure of PCR include a method described, for example, in Method in Molecular Biology, (31), 1994, 97-112. Another example includes a method described in WO 0009682.

Herein, the “sequence identity” refers to identity between two nucleotide sequences or two amino acids. The “sequence identity” is determined by comparing two sequences which are aligned in the optimal state over an all region of sequences to be compared. Herein, in optimal alignment of nucleotide sequences or amino acid sequences to be compared, addition or deletion (e.g. gap and the like) may be permitted. The sequence identity can be calculated by performing homology analysis to produce alignment using a program such as FASTA [Pearson & Lipman, Proc. Natl. Acad. Sol. USA, 4, 2444-2448 (1988)], BLAST [Altschul at al., Journal of Molecular Biology, 215, 403-410 (1990)], CLUSTAL W [Thompson, Higgins & Gibson, Nucleic Acid Research, 22, 4673-4680 (1994a)] and the like. The program is generally available at the website (http://www.ddbj.nig.ac.jp) of DNA Data Bank of Japan [International DNA Data Bank managed in National Institute of Genetics, Center for Information Biology and DNA Data Bank of Japan; CIB/DDBJ]. Alternatively, sequence identity can be also obtained using a commercially available sequence analyzing software. Specifically, for example, sequence identity can be calculated by performing homology analysis using GENETYX-WIN Ver. 5 (manufactured by Software Development Co. Ltd.) by a Lipman-Pearson method [Lipman, D. J. and Pearson, W. R., Science, 227, 1435-1441, (1985)] and producing alignment.

Examples of the “stringent condition” described in (f) include condition under which, in hybridization performed according to a conventional method described in Sambrook J., Frisch E. F., Maniatis T., Molecular Cloning 2nd edition, Cold Spring Harbor Laboratory press, for example, a hybrid is formed at 45° C. in a solution containing 6×SSC (a solution containing 1.5 m NaCl and 0.15 m trisodium citrate is 10×SSC) and, thereafter, this is washed with 2×SSC at 50° C. (Molecular Biology, John Wiley & Sons, N.Y. (1989), 6.3.1-6.3.6). A salt concentration in a washing step can be selected from condition from 2×SSC (low stringent condition) to 0.2×SSC (high stringent condition). A temperature in a washing step can be selected, for example, from condition from room temperature (low stringent condition) to 55° C. (high stringent condition). Alternatively, both of a salt concentration and a temperature can be changed.

A protein described in (h) indicates a c-Jun NH₂-terminal kinase presents in a cotton aphid among an insect c-Jun NH₂-terminal kinase, and includes a protein comprising an amino acid sequence described in (a).

In addition, a protein of the group A includes a protein comprising an amino acid sequence of an insect c-Jun NH₂-terminal kinase described in (g) and, more preferably includes, a protein in which when aligned with the amino acid sequence of SEQ ID NO: 1 so that maximum sequence identity is obtained, amino acid residues at positions corresponding to (I) position 64, (II) position 66, (III) position 90, (IV) position 107, (V) position 112, (VI) position 195, (VII) position 288, (VIII) position 289, (IX) position 358, (X) position 359 and (XI) position 368 of the amino acid sequence of SEQ ID NO: 1 are (I) threonine (at position corresponding to 64), (II) glutamine (at position corresponding to 66), (III) phenylalanine (at position corresponding to 90), (IV) Alanine (at position corresponding to 107), (V) arginine (at position corresponding to 112), (VI) threonine (at position corresponding to 195), (VII) Aspartic acid (at position corresponding to 288), (VIII) arginine (at position corresponding to 289), (IX) serine (at position corresponding to 358), (X) valine (at position corresponding to 359) and (XI) glutamine (at position corresponding to 368), respectively. Herein, the “aligned with the amino acid sequence of SEQ ID NO: 1 so that maximum sequence identity is obtained” means that sequence identity of a plurality of amino acid sequences to be analyzed Including the amino acid sequence of SEQ ID NO: 1 is analyzed by a program such as FASTA, BLAST, CLUSTAL W described above, and they are aligned. By aligning a plurality of sequences by the method, positions of conserved amino acid residues in each amino acid sequence can be determined regardless of insertion or deletion in an amino acid sequence. It is thought that conserved amino acid residues are present at the same position in the three-dimensional structure of the proteins of interest, and it is presumed that similar effect is possessed regarding specific function of the proteins of interest. For example, when insect c-Jun NH₂-terminal kinase including the c-Jun NH₂-terminal kinase sequence of which is disclosed in the present invention, are aligned with the amino acid sequence of SEQ ID NO: 1 so that maximum sequence identity of amino acid sequences is obtained, it is shown that amino acid residues at positions corresponding to (I) position 64, (II) position 66, (III) position 90, (IV) position 107, (V) position 112, (VI) position 195, (VII) position 288, (VIII) position 289, (IX) position 358, (X) position 359 and (XI) position 368 of the amino acid sequence of SEQ ID NO: 1 are (I) threonine (at position corresponding to 64), (II) glutamine (at position corresponding to 66), (III) phenylalanine (at position corresponding to 90), (IV) Alanine (at position corresponding to 107), (V) arginine (at position corresponding to 112), (Vi) threonine (at position corresponding to 195), (VII) Aspartic acid (at position corresponding to 288), (VIII) arginine (at position corresponding to 289), (IX) serine (at position corresponding to 358), (X) valine (at position corresponding to 359) and (XI) glutamine (at position corresponding to 368), respectively.

A substance having a pesticidal ability can be screened by using a method of assaying a pesticidal ability by measuring a pesticidal ability or controlling effect on the aforementioned pests.

Alternatively, a substance having a pesticidal ability can be also screened by the method of assaying a pesticidal ability using a c-Jun NH₂-terminal kinase. Specifically, when it has been identified that a pesticidal ability of a test substance is a certain value or more, or a certain value or less using the method of assaying a pesticidal ability using a c-Jun NH₂-terminal kinase, a substance having a pesticidal ability can be screened by selecting the substance.

Since a substance selected by the screening method has a pesticidal ability, it can be used as a pesticidal agent containing the substance or an agriculturally acceptable salt as an active ingredient.

Control of pests can be usually performed by application an effective amount of a pesticidal agent to a crop protected, a pest, or a habitat of a pest.

When a pesticidal agent is used for agriculture and forestry, its application amount is usually 0.1 to 1000 g in terms of an amount of a pesticidal agent per 1000 m². When a pesticidal agent is formulated into an emulsion, a water-dispersible powder, a flowable preparation, a microcapsule preparation or the like, the agent is usually applied by diluting with water to an active ingredient concentration of 1 to 10,000 ppm, and spraying this and, when a pesticidal agent is formulated into a granule, a powder or the like, the agent is usually applied as it is.

A pesticidal agent can be used by foliage-treating a plant such as a crop and the like which should be protected from pests, and can be also used by treating a seedbed before a plantlet of a Crop is transplanted, or a planting hole or a strain base at planting. Further, for the purpose of controlling pests habiting a soil of a cultivating land, the agent may be used by treating the soil. Alternatively, the agent may be used by a method of winding a resin preparation which has been processed to a sheet or a string, on a crop, stretching the preparation near a crop and/or spreading on a soil surface of a strain base.

When a pesticidal agent is used as a pest controlling agent for preventing en epidemic, an emulsion, a water-dispersible powder, a flowable or the like is usually applied by diluting with water so that an active ingredient concentration becomes 0.01 to 10,000 ppm, and an oily agent, an aerosol, a fumigant, a poison bait or the like is applied as it is.

Examples of one utility of a pesticidal agent include control of an external parasite of a livestock such as cattle, sheep, goat, and chicken, or a small animal such as dog, cat, rat, and mouse, in this case, the agent can be administered to an animal by the veterinarily known method. As a specific administration method, when systemic control is intended, the agent is administered, for example, by a tablet, mixing in feed, suppository, injection (intramuscular, subcutaneous, intravenous, intraperitoneal etc.) and the like, when non-systemic control is intended, the agent is used by a method of spraying an oily agent or an aqueous liquid agent, performing pour on or spot on treatment, washing an animal with a shampoo preparation or attaching a resin preparation which has been processed into a necklace or a ear tag to an animal. An amount of a pesticidal agent when administered to an animal body is usually in a range of 0.1 to 1,000 mg as expressed by total amount of a compound A and a compound B per 1 kg of an animal.

An application amount and an application concentration of them are both different depending on the situations such as a kind of a preparation, an application time, an application place, an application method, a kind of a pest, a damage degree and the like, can be increased or decreased regardless of the aforementioned range, and can be appropriately selected.

The aforementioned pesticidal agent can be used in the method of controlling pests as described above.

In addition, on the other hand, a pest can be also controlled by identifying a substance having a pesticidal ability evaluated by the aforementioned method of assaying a pesticidal ability possessed by a pest substance, having a first step and a second step using a c-Jun NH₂-terminal kinase selected from group A, and contacting the identified substance having a pesticidal ability with a pest. Herein, as a method of contacting an identified substance having a pesticidal ability with a pest, the aforementioned preparation method, application method and the like can be used.

An amino acid sequence shown in the group B is an amino acid sequence of insect c-Jun NH₂-terminal kinase comprising any amino acid sequence of the following (a) to (g).

(a) the amino acid sequence of SEQ ID NO: 1;

(b) an amino acid sequence with deletion, addition or substitution of one or more amino acids in the amino acid sequence of SEQ ID NO: 1, wherein said amino acid sequence has c-Jun NH₂-terminal kinase activity;

(c) an amino acid sequence that has sequence identity of 95% or more to the amino acid sequence of SEQ ID NO: 1, wherein said amino acid sequence has c-Jun NH₂-terminal kinase activity:

(d) the amino acid sequence encoded by the nucleotide sequence of SEQ ID NO: 2;

(e) an amino acid sequence encoded by a nucleotide sequence that has sequence identity of 75% or more to the nucleotide sequence of SEQ ID NO: 2, wherein said amino acid sequence has c-Jun NH₂-terminal kinase activity;

(f) an amino acid sequence encoded by a polynucleotide, wherein said polynucleotide hybridizes under a stringent condition to a polynucleotide comprising a nucleotide sequence complementary to the nucleotide sequence of SEQ ID NO: 2, wherein said amino acid sequence has c-Jun NH₂-terminal kinase activity; and

(g) an amino acid sequence of a cotton aphid c-Jun NH₂-terminal kinase.

Among amino acid sequences of the group B, a difference which can be recognized between an amino acid sequence represented by (a) and amino acid sequences represented by (b), (c), (e), (f) and (g) is deletion, substitution, addition or the like of a part of amino acids. These include, for example, deletion due to processing which the protein having an amino acid sequence represented by (a) undergoes in a cell. In addition, examples include deletion, substitution, addition and the like of an amino acid generated by naturally occurring gene mutation due to a spices difference or an individual difference of an organism from which the protein is derived, or gene mutation which is artificially introduced by a site-directed mutagenesis, a random mutagenesis, mutation treatment or the like.

The number of amino acids undergoing the deletion, substitution, addition or the like may be the number in a range that the peptidase activity of a c-Jun NH₂-terminal kinase can be found out. In addition, examples of substitution of an amino acid include substitution with an amino acid which is similar in characteristic in hydrophobicity, charge, pH and steric structure. Specific examples of the substitution include substitution in an group of (1) glycine, alanine; (2) valine, isoleucine, leucine; (3) aspartic acid, glutamic acid, asparagine, glutamine, (4) serine, threonine; (5) lysine, arginine; (6) phenylalanine, tyrosine and the like.

Examples of a procedure of artificially introducing the deletion, addition or substitution of an amino acid (hereinafter, collectively referred to as alteration of amino acid in some cases) include a procedure of introducing site-directed mutation into a DNA encoding an amino acid sequence represented by (a) and, thereafter, expressing this DNA by a conventional method. Herein, examples of a site-directed mutagenesis include a method utilizing amber mutation (gapped duplex method, Nucleic Acids Res., 12, 9441-9456 (1984)), a method by PCR using primers for mutation introduction, and the like. In addition, examples of a procedure of artificially altering an amino acid include a procedure of randomly introducing mutation into a DNA encoding an amino acid sequence represented by (a) and, thereafter, expressing this DNA by a conventional method. Herein, examples of a method of randomly introducing mutation include a method of performing PCR using a DNA encoding any of the aforementioned amino acid sequences as a template, and using a primer pair which can amplify each full length DNA at reaction condition under which an addition amount of each of dATP, dTTP, dGTP and dCTP used as a substrate is changed from a conventional concentration, or at reaction condition under which a concentration of Mg²⁺ promoting a polymerase reaction is increased from a conventional concentration. Examples of the procedure of PCR include a method described, for example, in Method in Molecular Biology, (31), 1994, 97-112. Another example includes a method described in WO 0009682.

Herein, the “sequence identity” refers to identity between two nucleotide sequences or two amino acids. The “sequence identity” is determined by comparing two sequences which are aligned in the optimal state over an all region of sequences to be compared. Herein, in optimal alignment of nucleotide sequences or amino acid sequences to be compared, addition or deletion (e.g. gap and the like) may be permitted. The sequence identity can be calculated by performing homology analysis to produce alignment using a program such as FASTA [Pearson & Lipman, Proc. Natl. Acad. USA, 4, 2444-2448 (1988)], BLAST Altschul et al. Journal of Molecular Biology, 215, 403-410 (1990)), CLUSTAL W [Thompson, Higgins & Gibson, Nucleic Auld Research, 22, 4673-4680 (1994a)] and the like. The program is generally available at the website (http://www.ddbj.nig.ac.jp) of DNA Data Bank of Japan [International DNA Data Bank managed in National Institute of Genetics, Center for Information Biology and DNA Data Bank of Japan; CIB/DDBJ]. Alternatively, sequence identity can be also obtained using a commercially available sequence analyzing software. Specifically, for example, sequence identity can be calculated by performing homology analysis using GENETYX-WIN Ver. 5 (manufactured by Software Development Co. Ltd.) by a Lipman-Pearson method [Lipman, D. J. and Pearson, W. R., Science, 227, 1435-1441, (1985)] and producing alignment.

Examples of the “stringent condition” described in (f) include condition under which, in hybridization performed according to a conventional method described in Sambrook J., Frisch E. F., Maniatis T., Molecular Cloning 2nd edition, Cold Spring Harbor Laboratory press, for example, a hybrid is formed at 45° C. in a solution containing 6×SSC (a solution containing 1.5 m NaCl and 0.15 m trisodium citrate is 10×SSC) and, thereafter, this is washed with 2×SSC at 50° C. (Molecular Biology, John Wiley & Sons, N.Y. (1989), 6.3.1-6.3.6). A salt concentration in a washing step can be selected from condition from 2×SSC (low stringent condition) to 0.2×SSC (high stringent condition). A temperature in a washing step can be selected, for example, from condition from room temperature (low stringent condition) to 65° C. (high stringent condition). Alternatively, both of a salt concentration and a temperature can be changed.

A protein having an amino acid sequence described in (g) indicates a c-Jun NH₂-terminal kinase presents in a cotton aphid among an insect c-Jun NH₂-terminal kinase, and includes a protein comprising an amino acid sequence described in (a).

A protein having an amino acid sequence shown in the group B can be prepared, for example, according to a method described later using a polynucleotide encoding an amino acid sequence shown in the group B.

An insect c-Jun NH₂-terminal kinase can be used as a reagent that provides an indicator to evaluate a pesticidal activity. Specifically, for example, an insect c-Jun NH₂-terminal kinase can be used as a reagent that provides an indicator to evaluate a pesticidal activity by using as a c-Jun NH₂-terminal kinase used in the method of assaying a pesticidal ability using a c-Jun NH₂-terminal kinase. In addition, a more specific method can be performed according to the aforementioned method of measuring the activity of a c-Jun NH₂-terminal kinase.

In addition, when an insect c-Jun NH₂-terminal kinase is used as a reagent that provides an indicator to evaluate a pesticidal activity, more preferably, it is desirable that an insect c-Jun NH₂-terminal kinase is a c-Jun NH₂-terminal kinase having an amino acid sequence shown in the group B.

A polynucleotide having a nucleotide sequence encoding an amino acid sequence shown in the group B (hereinafter, referred to as polynucleotide group B in some cases) has a nucleotide sequence from which a protein having an amino acid sequence can be produced shown in the group B, in a cell of an organism or an in vitro translation system. A polynucleotide group B may be a DNA cloned from a nature, a DNA in which deletion, substitution or addition of a nucleotide is introduced into a DNA cloned from a nature, for example, by a site-directed mutagenesis or a random mutagenesis, or an artificially synthesized DNA. Specifically, examples include a polynucleotide comprising a nucleotide sequence represented by SEQ ID NO: 2.

### <First Obtaining Method>

For example, a method of obtaining a polynucleotide comprising the nucleotide sequence of SEQ ID NO: 2 included in the polynucleotide group B will be shown below. As a step, total RNA is obtained from cotton aphids, cDNA library is synthesized, and PCR amplification is performed, thereby, a polynucleotide of interest can be obtained.

A population of adults and larvae of Aphis gossypii, which have been reared on leaves of potted cucumber, is scraped from the surface of the leaves with a small brush, and 630 mg of the obtained population is crushed into a powder in liquid nitrogen using a mortar and a pestle. From the resulting frozen crushed powder, RNA is isolated using a RNA extracting reagent ISOGEN (manufactured by Nippon Gene) as follows. After 10 ml of ISOGEN is added to the frozen crushed powder in the mortar, the crushed powder is ground for 10 minutes while kept on ice. After grinding, a fluid sample is transferred to a 15 ml tube with a pipette, and 2 ml of chloroform (manufactured by Wako Pure Chemical Industries, Ltd.) is added thereto. Immediately, the mixture is vigorously shaken for 15 seconds and then left at rest at room temperature for 3 minutes. Then, the resulting mixture is centrifuged at 12,000×g at 4° C. for 15 minutes, and each 5 ml of aqueous layer are transferred to two new tubes. After 5 ml of ISOGEN is added to each tube, the mixture was immediately shaken vigorously for 15 seconds, and left at rest at room temperature for 3 minutes. Then, the resulting mixture is centrifuged at 12,000×g at 4° C. for 15 minutes, and each 10 ml of aqueous layer are transferred to new 50 ml tubes, respectively. Subsequently, 10 ml of isopropanol (manufactured by Wako Pure Chemical Industries, Ltd.) is added to each tube, and the mixture is kept on ice for 30 minutes. The resulting mixture is centrifuged at 12,000×g at 4° C. for 10 minutes to precipitate RNA. After the supernatant is removed, 20 ml of 70% ethanol is added to the residue. The resulting mixture is centrifuged at 10,000×g at 4° C. for 5 minutes. After the supernatant is removed, the precipitate of total RNA is slightly dried and then dissolved in 1 ml of commercially available RNase-free water (Nacalai Tesque Inc.). An absorbance of the prepared total RNA is measured at 260 nm to calculate a concentration according to a conventional method.

RT-PCR is performed employing total RNA of cotton aphid obtained by the aforementioned method as a template, and using random primers (manufactured by Invitrogen) and superscript III (manufactured by Invitrogen) according to the manual annexed to the reagent, to synthesized a cDNA library.

PCR is performed employing cDNA library of cotton aphid obtained by the aforementioned method as a template, and using an oligonucleotide primer comprising the nucleotide sequence of SEQ ID NO: 3 and an oligonucleotide primer comprising the nucleotide sequence of SEQ ID NO: 4 as well as a Pfu Turbo (manufactured by Stratagene) according to the manual annexed to the reagent. The conditions of the PCR are as follows: incubation at 94° C. for 10 minutes; followed by 30 cycles of PCR, one cycle being 94° C. for 30 seconds, 50° C. for 30 seconds and 72° C. for 2 minutes; and followed by incubation at 72° C. for 7 minutes.

As described above, a polynucleotide comprising the nucleotide sequence of SEQ ID NO: 2 can be obtained.

### <Second Obtaining Method>

Alternatively, a polynucleotide shown in the polynucleotide group B can be also obtained by preparing a polynucleotide with mutation introduced therein by a method utilizing amber mutation which is the aforementioned site-directed mutagenesis, a method by PCR using a primer for introducing mutation or the like, using as a template a polynucleotide comprising the nucleotide sequence of SEQ ID NO: 2.

### <Third Obtaining Method>

Alternatively, a polynucleotide shown in the polynucleotide group B can be also obtained by a hybridization method using a polynucleotide comprising the nucleotide sequence of SEQ ID NO: 2 as a probe. More specifically, the third obtaining method can be performed according to a conventional hybridization described in the aforementioned Sambrook J., Frisch E. F., Maniatis T., Molecular Cloning 2nd edition, published by Cold Spring Harbor Laboratory press.

### <Fourth Obtaining Method>

Alternatively, a polynucleotide shown in the polynucleotide group B can be also obtained by preparing a primer based on an amino acid sequence of the known insect c-Jun NH₂-terminal kinase and performing PCR. For isolation of homologues of c-Jun NH₂-terminal kinase gene from other insect species such as German cockroach germanica), degenerate primers are designed using Codehop program (publicly accessible on the website of Blocks Protein Analysis Server operated within the Fred Hutchinson Cancer Research Center at http://blocks.fhcrc.org/blocks/codehop.html), and based on the sequence of the aforementioned cotton aphid-derived c-Jun NH₂-terminal kinase gene and the previously-known nucleotide sequences of Ancylostoma caninum (NCBI accession number AAF00539), Xenopus laevis (BAB91438, BAB85483), Homo sapiens (NP_{—}620448. NP_{—}002744), Mus musculus (BAC31240), Rattus norvegicus ($43969), Gallus genus (BAA19188), Drosophila melanogaster (AAF52883, AAC47325), Anopheles gambiae (EAA05905) and Aedes albopictus(AA031950).

Partial sequences of a homologue of c-Jun NH₂-terminal kinase gene in a selected insect species are amplified by a series of PCR using first-strand cDNA derived from the insect species as a template. Herein, the first-strand cDNA as a template is prepared by the aforementioned method using Superscript III. Amplification by PCR is performed using a set of degenerate primers as a forward primer and a reverse primer as well as Amplitaq Gold (manufactured by Applied Biosystems) according to the manufacturer's procedure annexed to the reagent. PCR conditions are 94° C. for 10 minutes; followed by 40 cycles of PCR, one cycle being 94° C. for 30 seconds, 45° C. for 1 minute, and 72° C. for 1 minute per 1 kb of a length of a predicted amplification product; and followed by 72° C. for 7 minutes. The PCR product is analyzed and purified by agarose gel electrophoresis to obtain DNA of interest. Further, the obtained DNA is cloned into the pCR-XL-TOPO vector (manufactured by Invitrogen), and sequenced.

Then, primers specific for the resulting partial sequences of the insect homologue of c-Jun NH₂-terminal kinase gene are designed, and 3′RACE PCR or 5′RACE PCR is performed in order to obtain a full-length sequence of the gene. 3′ and 5′ RACE PCRs are, performed employing first-strand cDNA prepared from the insect total RNA as a template and using SMART PCR cDNA Synthesis Kit (manufactured by Clontech) according to the manufacturer's instructions annexed to the kit.

In 3′RACE and 5′RACE reactions, universal primer mix (UPM) contained in SMART PCR cDNA Synthesis Kit is used in combination with a forward primer or a reverse primer which is specific for the sequence of interest. PCR conditions are 1 cycle at 94° C. for 10 minutes; followed by 40 cycles of PCR, one cycle being 94° C. for 15 seconds, 63° C. for 15 seconds, and 72° C. for 1 minute per 1 kb of a length of a predicted amplification product; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

When a distinct amplification product is not obtained by the first-round PCR, nested PCR is performed using the first-round PCR product as a template. As primers, NUP primer contained in SMART PCR cDNA Synthesis Kit is used in combination with a specific forward primer or a specific reverse primer which is designed to bind internal sequence of the first-round PCR product of interest. PCR conditions are 1 cycle at 94° C. for 10 minutes; followed by 40 cycles of PCR, one cycle being 94° C. for 15 seconds, 63° C. for 15 seconds, and 72° C. for 2 minutes; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

The above sequencing results reveal 5′-terminal sequence and 3′-terminal sequence, each encoding N-terminal region and C-terminal region of the insect c-Jun NH₂-terminal kinase, respectively.

Thus, a polynucleotide shown in the polynucleotide group B can be obtained by PCR by preparing a primer based on an amino acid sequence of the known insect c-Jun NH₂-terminal kinase.

A polynucleotide having a nucleotide sequence complementary to a polynucleotide sequence of the polynucleotide group B can be used for obtaining a polynucleotide shown in the polynucleotide group B using a hybridization method.

The obtaining method in the present invention comprises a step of detecting a desired polynucleotide by hybridization, a step of identifying the detected desired polynucleotide, and a step of recovering the identified desired polynucleotide. Each step will be explained specifically below.

A step of detecting a desired polynucleotide by hybridization, and a step of identifying the detected desired polynucleotide can be performed by using, as a probe, a polynucleotide having a nucleotide sequence having complementarity to a nucleotide sequence of a polynucleotide group B, according to the method described, for example, in “Molecular Cloning: A Laboratory Manual 2nd edition” (1989), Cold Spring Harbor Laboratory Press, “Current Protocols In Molecular Biology” (1987), John Wiley & Sons, Inc. ISBN0-471-50338-X and the like.

Specifically, for example, a DNA having a nucleotide sequence having complementarity to a nucleotide sequence represented by SEQ ID NO: 2 is labeled with a radioisotope or a fluorescently labeled by the known method using Random Primed DNA Labelling Kit (manufactured by Boehringer), Random Primer DNA Labelling Kit Ver. 2 (manufactured by TAKARA SHUZO Co., Ltd.), ECL Direct Nucleic Acid Labelling and Detection System (manufactured by Amersham Biosciences), or Megaprime DNA-labelling system (manufactured by Amersham Biosciences), and this can be used as probe.

Examples of condition for hybridization include stringent condition, and specifically, examples include condition under which incubation is performed at 65° C. in the presence of 6×SSC (0.9M NaCl, 0.09M sodium citrate), a 5×Denhart's solution (0.1% (w/v) Ficoll 400, 0.1% (w/v) polyvinylpyrrolidone, 0.1% BSA), 0.5% (w/v) SDS and 100 μg/ml denatured salmon spermatozoon DNA, or in a DIG EASY Hby solution (Boehringer Mannheim) containing 100 μg/ml denatured salmon spermatozoon DNA, then, incubation is performed two times at room temperature for 15 minutes in the presence of 1×SSC (0.15 m NaCl, 0.015 m sodium citrate) and 0.5% SDFS and, further, incubation is performed at 68° C. for 30 minutes in the presence of 0.1×SSC (0.015 m NaCl, 0.0015 m sodium citrate) and 0.5% SDS. More specifically, for example, a probe labeled with ³²P can be made by employing a polynucleotide having a nucleotide sequence having complementarity to a nucleotide sequence of a polynucleotide group B as a template, using Megaprime DNA-labelling system (manufactured by Amersham Pharmacia Biotech) and using a reaction solution designated in a kit. Colony hybridization is performed using this probe according to a conventional method, incubation is performed at 65° C. in the presence of 6×SSC (0.9M NaCl, 0.09M sodium citrate), a 5×Denhart's solution (0.1% (w/v) Picoll 400, 0.1% (w/v) polyvinylpyrrolidone, 0.1% BSA), 0.5% (w/v) SDS and 100 μg/ml denatured salmon spermatozoon DNA, or in a DIG EASY Hyb solution (Boehringer Mannheim) containing 100 μg/ml denatured salmon spermatozoon DNA, then, incubation is performed two times at room temperature for 15 minutes in the presence of 1×SSC (0.15 m NaCl, 0.015 m sodium citrate) and 0.5% SDS and, further, incubation is performed at 68° C. for 30 minutes in the presence of 0.1×SSC (0.015 NaCl, 0.0015 m sodium citrate) and 0.5% SDS, thereby, (a colony containing) a hybridizing polynucleotide can be detected. Thus, a desired polynucleotide can be detected by hybridization, and the detected desired polynucleotide can be identified.

For recovering the identified desired polynucleotide, a plasmid DNA can be recovered from a colony containing the polynucleotide detected and identified by the aforementioned method, for example, according to a method such as the alkali method described in “Molecular Cloning: A Laboratory Manual 2nd edition” (1989), Cold Spring Harbor Laboratory Press. A nucleotide sequence of the recovered desired polynucleotide (plasmid DNA) can be confirmed by a Maxam Gilbert method (described, for example, in Maxam, A. M & W Gilbert, Proc. Natl. Aced. Sci. USA, 74, 560, 1977 etc.) or a Sanger method (described, for example, in Sanger, F. & A. R. Coulson, J. Mol. Biol., 94, 441, 1975 Sanger, F. & Nicklen and A. R. Coulson., Proc. Natl. Acad. Sci. USA, 74, 5463, 1977 etc.). Thereupon, for example, commercially available Termo Sequenase II dye terminator cycle sequencing kit (manufactured by Amersham biosciences), Dye Terminator Cycle Sequencing FS Ready Reaction Kit (manufactured by Applied Biosystems) and the like can be used.

A polynucleotide having a partial nucleotide sequence of a nucleotide sequence of the polynucleotide group B or a nucleotide sequence complementary to the partial nucleotide sequence can be used for obtaining a polynucleotide shown in the polynucleotide group B using PCR. More specifically, examples include a polynucleotide comprising a nucleotide sequence represented by SEQ ID NO: 3 or 4. The obtaining method in the present invention includes a step of amplifying a desired polynucleotide by PCR, a step of identifying the amplified desired polynucleotide, and a step of recovering the identified desired polynucleotide. Each step will be specifically explained below.

In a step of amplifying a desired polynucleotide by PCR, specifically, a DNA designed and synthesized from a partial nucleotide sequence of a nucleotide sequence of a polynucleotide group B or a nucleotide sequence having complementarity to the partial nucleotide sequence, based on an about 20 bp to about 40 bp nucleotide sequence, for example, a nucleotide sequence selected from a nucleotide sequence represented by SEQ ID NO: 2 and a sequence having complementarity to a nucleotide sequence represented by SEQ ID NO: 2 can be used as a primer set. Examples of a primer set include a set of a polynucleotide comprising a nucleotide sequence represented by SEQ ID NO: 3 and a polynucleotide comprising a nucleotide sequence represented by SEQ ID NO: 4. A PCR reaction solution is prepared, for example, by adding a reaction solution designated by a commercially available PCR kit to a cDNA library prepared by the aforementioned method. Reaction condition can be changed depending on a primer set to be used, and for example, condition under which after incubation at 94° C. for 10 seconds, around 40 cycles is repeated, 1 cycle being 94° C. for 15 seconds, 60° C. for 15 seconds, and 72° C. for 3 minutes and, further, incubation is performed at 72° C. for 3 minutes, condition under which incubation is performed at 94° C. for 2 minutes, thereafter, incubation is performed at about 8° C. for 3 minutes and, thereafter, around 40 cycles is repeated, 1 cycle being 94° C. for 30 seconds, 55° C. for 30 seconds, and 72° C. for 4 minutes, or condition under which 5 to 10 cycles is performed, 1 cycle being incubation at 94° C. for 5 seconds and, then, 72° C. for 4 minutes and, further, around 20 to 40 cycles is performed, 1 cycle being incubation at 94° C. for 5 seconds and, then, 70° C. for 4 minutes, can be used. In the PCR, for example, PfuUltra High Fidelity polymerase (manufactured by Stratagene), Amplitaq Gold (manufactured by Applied Biosystems), Takara Heraculase (Trademark) (manufactured by TAKARA SHUZO Co., Ltd.), a DNA polymerase contained in Advantage cDNA PCR Kit (manufactured by Clonetech), TaKaRa Ex Tag (manufactured by TAKARA SHUZO Co., Ltd.), PLATINUM™ PCR SUPER Mix (manufactured by Lifetech Oriental) can be used.

Identification of a desired polynucleotide amplified by PCR can be performed by measuring a molecular weight by agarose gel electrophoresis according to the method described in “Molecular Cloning: A Laboratory Manual 2nd edition” (1989), Cold Spring Harbor Laboratory Press. In addition, regarding the amplified desired polynucleotide, a sequencing reaction is performed using a commercially available DNA sequencing reaction kit, for example, Dye Terminator Cycle Sequencing FS Ready Reaction Kit (manufactured by Applied Biosystems) according to a manual annexed to the kit, and the nucleotide is analyzed using a DNA sequencer 3100 (manufactured by Applied Biosystems), thereby, a nucleotide sequence of the amplification fragment can be read.

Examples of a method of recovering the identified desired polynucleotide include a method of purifying and recovering the aforementioned polynucleotide identified by agarose gel electrophoresis from an agarose gel according to the method described in “Molecular Cloning: A Laboratory Manual 2nd edition” (1989), Cold Spring Harbor Laboratory Press. In addition, the thus recovered polynucleotide or a desired polynucleotide amplified by PCR can be cloned into a vector according to a conventional method described in “Molecular Cloning: A Laboratory Manual 2nd edition” (1989), Cold Spring Harbor Laboratory Press, and “Current Protocols In Molecular Biology” (1987), John Wiley & Sons, Inc. ISBN0-471-50338-X. Examples of a vector to be used include pUCA119 (manufactured by TAKARA SHUZO Co., Ltd.), pTVA118N (manufactured by TAKARA SHUZO Co., Ltd.), pBluescriptII (manufactured by Toyobo Co., Ltd.), pCR2.1-TOPO (manufactured by Invitrogen) and the like. In addition, a nucleotide sequence of the cloned polynucleotide can be confirmed by a Maxam Gilbert method (described, for example, in Maxam, A. M &W. Gilbert, Proc. Natl. Acad. Sci. USA, 74, 560, 1977) or a Sanger method (described, for example, in Sanger, F. & A. R. Coulson, J. Mol. Biol., 94, 441, 1975, Sanger, F, & Nicklen and A. R. Coulson., Prod. Natl. Acad. Sci. USA, 74, 5463, 1977). Thereupon, for example, a commercially available Termo Sequenase II dye terminator cycle sequencing kit (manufactured by Amersham biosciences), Dye Terminator Cycle Sequencing FS Ready Reaction Kit (manufactured by Applied Biosystems) and the like can be used.

In addition, a polynucleotide having a partial nucleotide sequence of a nucleotide sequence of the polynucleotide group B or a nucleotide sequence complementary to the partial nucleotide sequence can be used for obtaining a polynucleotide shown in the polynucleotide group B using not only a PCR method, but also the aforementioned hybridization method. More specifically, examples include a polynucleotide comprising a nucleotide sequence represented by SEQ ID NO: 3 or 4.

Examples of a method for preparing a protein having an amino acid sequence shown in the group B include a method of culturing a transformant with a polynucleotide selected from a polynucleotide group B introduced therein, and recovering the produced protein. In addition, for preparing a transformant used herein, it is a work such as preparation of a circular polynucleotide containing a polynucleotide in which a polynucleotide selected from a polynucleotide group B is operably ligated to a baculovirus-derived promoter. The method will be explained in detail below.

In addition, a c-Jun NH₂-terminal kinase shown in a group A which is used in the method of assaying a pesticidal ability using a c-Jun NH₂-terminal kinase can be prepared and obtained by the similar method, using a polynucleotide having a nucleotide sequence encoding a c-Jun NH₂-terminal kinase used.

Baculovirus is a virus belonging to a diverse group of large double-stranded DNA viruses that infect many different species of insects as their natural hosts. The baculovirus genome is replicated and transcribed in the nuclei of infected host cells where the large circular baculovirus DNA (between 80 and 200 kb) is packaged into rod-shaped nucleocapsids. Examples of isolates used in foreign gene expression are Autographa californica multiple nuclear polyhedrosis virus (AcMNPV) and Bombyx mori (silkworm) nuclear polyhedrosis virus (BmNPV).

A baculovirus-derived promoter means a promoter of a gene contained in a baculovirus genome. Among them, examples of a promoter of baculovirus used for expressing a foreign gene include a promoter of a polyhedrin gene, and a promoter of a p10 gene (Harris and Polayes (1997). Focus 19, 6-8).

A promoter of a polyhedrin (polyhedron) gene is a promoter of a gene encoding polyhedrin which is a main component of an intranuclear inclusion body produced when baculovirus infects an insect cell. Polyhedrin is not a protein necessary for replicating a virus and, by substituting its gene with a gene of a protein of interest, the protein of interest amounting to 50% of a cell protein may be expressed.

In the present invention, “operably linked” means that a polynucleotide containing a gene of interest is linked downstream of a polynucleotide containing a promoter sequence so that the gene of interest can be transcribed in a used transcription system. Specifically, for example, when a promoter of a polyhedrin gene described later is used, a polynucleotide containing a gene of interest may be linked downstream of a promoter of a polyhedrin gene. In addition, for example, when a promoter other than a polyhedrin gene promoter is used, it is also possible to link a polynucleotide containing a gene of interest downstream of a polynucleotide containing a promoter sequence other than a polyhedrin gene promoter. More specifically, for example, when a plasmid pFastbacHT (manufactured by Invitrogen) vector utilizing a polyhedrin gene promoter is used, the polynucleotide can be operably linked by ligating a gene of interest into a restriction enzyme site such as BamHI, EcoRI, SalI, SpeI, NotI, XbaI, PstI, XhoI, SphI, KpnI, and HindIII located downstream of a polyhedrin gene promoter.

In the present invention, the “circular polynucleotide” is a polynucleotide which has been made to be circular by binding of ends of the polynucleotide strand, and examples include chromosomal DNAs of many bacteria in addition to a plasmid DNA, a bacmid DNA and the like.

A plasmid DNA is a relatively low-molecular circular polynucleotide, and examples include pET (manufactured by Takara Mirus Bio Inc.) and pBluescriptII (manufactured by Stratagene), used for cloning and expression in E. coli. Additional examples include pFastBacl, pFastBac HT A, pFastBac HT B, pFastBac HT C, pFastBac Dual. pBlueBacII (manufactured by Invitrogen), pACSG2 (manufactured by Pharmingen) and the like, which contain a baculovirus expression cassette.

The bacmid is a high molecular weight DNA that consists of a BAC (bacterial artificial chromosome) that contains the entire baculoviral genome, for example bMON14272 (136 kb) that is present in DH10Bac™ E. coli cells (invitrogen). Bacmid DNA propagates as a large plasmid in E. coli cells and may contain an expression cassette for expression of a foreign gene under control of a baculoviral promoter.

A circular polynucleotide in which a polynucleotide comprising a nucleotide sequence encoding an amino acid sequence shown in the group B is operably linked to a baculovirus-derived promoter is specifically, for example, a circular polynucleotide containing a DNA comprising a cotton aphid c-Jun NH₂-terminal kinase gene operably linked to a polyhedrin promoter of baculovirus, and can be prepared and obtained, for example, according to the following method.

A plasmid DNA containing a cotton aphid c-Jun NH₂-terminal kinase gene cloned according to the aforementioned method is digested with EcoRI, and the resulting about 1.3 kbp DNA fragment comprising a cotton aphid c-Jun NH₂-terminal kinase gene is ligated with a plasmid vector pFastBac HT B (manufactured by Invitrogen) which has been digested with EcoRI in advance The obtained plasmid is one example of a circular polynucleotide containing a DNA comprising a cotton aphid c-Jun NH₂-terminal kinase gene operably linked to a polyhedrin promoter of baculovirus. In addition, according to the manual annexed to Bac-to-Bac Baculovirus Expression System (manufactured by invitrogen), this plasmid may be introduced into Escherichia coli DB10Bac, and a DNA containing a polyhedrin gene promoter and a cotton aphid c-Jun NH₂-terminal kinase gene can be inserted into a bacmid DNA by a recombination in the cell. For example, by the aforementioned method, a circular polynucleotide containing a DNA comprising a cotton aphid c-Jun NH₂-terminal kinase gene operably linked to a polyhedrin promoter of baculovirus can be prepared and obtained.

Similarly, a circular polynucleotide can be prepared by ligating a nucleotide encoding an amino acid sequence shown in the group B to a vector.

In the present invention, the “origin of replication” is the specific DNA sequence necessary for replicating itself in a host cell. Examples of origin of replication include colE1 and f1 for bacterial plasmids. In addition, a homologous repeated (hrs) region, and a non-hr region are present in a bacmid DNA (Pijlman et al. (2003) Journal of General Virology 84, 2669-2678).

One example of the circular polynucleotide is a baculovirus shuttle vector. Herein, a baculovirus shuttle vector means the bacmid DNA. The bacmid DNA can be propagated and genetically engineered in E. coli. Upon isolation from E. coli and introduction into an insect host cell, bacmids can be propagated as a virus. For example in the case of bMON14272 (invitrogen), the recombinant bacmids are generated in E. coli by transposition of a mini-Tn7 element, containing the baculoviral expression cassette from a pFastBac™ donor plasmid to the mini-attTn7 attachment site on the bacmid.

Insect cells to be used as host for the propagation of the baculovirus and for expression of the foreign protein by means of the recombinant baculovirus include cell lines derived from Spodoptera frugiperda or from Trichoplusia ni. Examples of such cell lines are, Sf21 cells, Sf9 cells, Tn-368 or High Five cells or Mimic Sf9 Insect cells (Invitrogen).

A transformant is a eukaryotic cell or a prokaryotic cell which has been genetically altered by introduction of a foreign polynucleotide into a cell. Examples of a transformant include an Escherichia coli cell transformed by introduction of a plasmid containing a baculovirus expression cassette such as plasmid vector pFastBac (manufactured by Invitrogen) and the like. In addition, examples of the technique of introducing a DNA into a host cell include transformation, transfection, protoplast fusion, lipofection, electroporation and the like.

Examples of a transformant in which a polynucleotide encoding an amino acid sequence shown in the group B is introduced include transformed Escherichia coli in which a DNA comprising a cotton aphid c-Jun NH₂-terminal kinase gene operably linked to a polyhedrin promoter of baculovirus is introduced. Specifically, the transformant can be prepared according to the following method.

A transformant can be prepared by introducing a plasmid vector pFastBac HT B (manufactured by Invitrogen) in which a DNA containing a cotton aphid c-Jun NH₂-terminal kinase gene is inserted in EcoRI site into an Escherichia coli cell according to the method described in “Molecular Cloning: A Laboratory Manual 2nd edition” (1989), Cold Spring Harbor Laboratory Press. In addition, a transformant can be also prepared by introducing the same plasmid vector into Escherichia coli DH10Bac according to a method described in a manual annexed to Bac-to-Bac Baculovirus Expression System (manufactured by Invitrogen).

Alternatively, a transformant can be also obtained by transfecting the bacmid DNA in which a fragment containing a polyhedrin gene promoter and a cotton aphid c-Jun NH₂-terminal kinase gene is inserted, into an insect cell according to a method described in a manual annexed to Bac-to-Bac Baculovirus Expression System (manufactured by Invitrogen).

A recombinant baculovirus is baculovirus in which the sequence of the baculovirus genome has been altered by genetic-engineering technique.

Specific examples of recombinant baculovirus include recombinant baculovirus containing a DNA fragment comprising a cotton aphid c-Jun NH₂-terminal kinase gene operably linked to a polyhedrin promoter of baculovirus A recombinant baculovirus can be prepared, for example, by homologous recombination between baculoviral DNA and transfer vector DNA in insect cells Mitts (1996) Cytotechnology 20, 111-123). Alternatively, recombinant baculovirus can be also prepared, for example, by introducing a recombinant bacmid DNA containing a DNA fragment comprising a cotton aphid c-Jun NH₂-terminal kinase gene operably linked to a polyhedrin promoter of baculovirus prepared by the aforementioned method into insect cells. Specifically, recombinant baculovirus can be prepared by transfecting a recombinant bacmid DNA containing a DNA fragment comprising a cotton aphid c-Jun NH₂-terminal kinase gene operably linked to the aforementioned polyhedrin promoter of baculovirus into an insect cell according to the method described in a manual annexed to Bac-to-Bac Baculovirus Expression System (manufactured by Invitrogen). More specifically, the recombinant bacmid DNA is transfected into Sf9 cells (ATCC: CRL-1711) according to the manual annexed to Bac-to-Bac Baculovirus (Trademark)) Expression System (manufactured by Invitrogen) to obtain recombinant baculovirus. For the transfection, cellfectin (manufactured by Invitrogen), Grace's Insect Cell Culture Medium supplemented with L-amino acids (manufactured by Invitrogen, Gibco), 10% Foetal Bovine Serum (manufactured by Clontech), and penicillin/streptomycin (manufactured by Life Technologies) are used. In addition, for transfection, 2 μg of bacmid DNA and 7 μl of cellfectin are used. A P1 recombinant baculovirus stock is recovered after 8 days according to the manual annexed to Bac-to-Bac Baculovirus (Trademark) Expression System (manufactured by) Invitrogen). For example, 0.5 ml of this baculovirus stock can be further propagated by inoculating on 300 ml of Sf9 cell cultures of 1×10⁶ cells/ml. The amplified baculovirus stock is harvested after 4 days according to the manual annexed to Bac-to-Bac Baculovirus (Trademark) Expression System (manufactured by Invitrogen). The Sf9 cell are suspension-cultured in Erlenmeyer flasks at 27° C. and at 135 rpm. A component of a medium used in this culturing include Grace's Insect Cell Culture Medium supplemented with L-amino acids (manufactured by Invitrogen, Gibco), 10% Foetal Bovine Serum (manufactured by Clontech), penicillin/streptomycin (manufactured by Life Technologies), and final consideration 0.1% Pluronic F-68 (manufactured by Sigma-aldrich).

A c-Jun NH₂-terminal kinase can be prepared by culturing a transformant prepared by the aforementioned method, and recovering the produced insect-derived c-Jun NH₂-terminal kinase.

C-Jun NH₂-terminal kinase protein may be produced by for example a recombinant baculovirus/Sf9 cell expression system. This system is one of the most powerful and versatile eukaryotic expression systems available, and may be used to express heterologous genes from many different sources, including fungi, plants., bacteria and viruses.

Alternatively, c-Jun NH₂-terminal kinase protein may be produced by a recombinant E. coli expression system. This system is the most frequently used prokaryotic expression system for the high-level production of heterologous proteins. E. coli is genetically and physiological the best characterized organism known, it is easy to manipulate, many tools are available, it is) able to grow very fast, it grows on cheap complex or well-defined minimal media and it has an extremely high capacity to synthetize heterologous protein.

In addition, an insect-derived c-Jun NH₂-terminal kinase produced by culturing a transformant is lysed by a method such as sonication, French press, and Dyno mill, and recovered in a form contained in a cell crude extract, and a purified protein can be obtained by using a procedure conventionally used in enzyme purification such as ion exchange column chromatography, reverse phase column chromatography, gel filtration column chromatography and the like. Alternatively, when it is devised that an insect-derived c-Jun NH₂-terminal kinase is produced in a form with His-tag, a purified protein can be obtained rapidly from a cell crude extract by affinity column chromatography which specifically recognizes and binds to the His-tag. By the method, an insect-derived c-Jun NH₂-terminal kinase can be prepared.

For example, an insect-derived c-Jun NH₂-terminal kinase can be prepared by culturing a transformed insect cell with a DNA fragment containing a cotton aphid c-Jun NH₂-terminal kinase gene operably linked to a polyhedrin promoter of baculovirus, and grinding the cell with a French press, followed by purification with column chromatography.

More specifically, for example, 4×10⁸ Sf9 cells are suspended in 30 ml of a stock solution of recombinant baculovirus containing a recombinant bacmid DNA containing a DNA fragment comprising a cotton aphid c-Jun NH₂-terminal kinase gene operably ligated to a polyhedrin promoter of baculovirus, the suspension is rotation-cultured in 125 ml Erlenmeyer at 135 rpm, at 27° C. for 1 hour. Thereafter, each ⅓ of the cell suspension is placed into three 25 ml Erlenmeyer flasks, a medium is added to each flask so that a volume of a culturing solution becomes 100 ml, 1 ml of a 10% Pluronic solution is added, and this is cultured again. After 48 hours, Sf9 cells infected with baculovirus are harvested by centrifugation at 290×g for 5 minutes. Buffer A (50 mM Hepes-HaOH pH7.5, 0.5 m NaCl, 10 mM imidazole) is added to the harvested Sf9 cells to suspend them, and cells are lysed at a pressure of 1, 500 psi using a French press (manufactured by Thermo Spectronic) according to a method described in the annexed manual. This French-pressed solution is centrifuged at 13,000×g at 4° C. for 20 minutes, and the resulting supernatant is filtered through a 0.45 mm filter., Then, this is injected into two HiTrap/HisTrap affinity columns (column volume 5 ml, manufactured by Amersham biosciences) connected in series which are equilibrated with buffer A (50 mM Hepes-HaOH pH7.5, 0.5 m NaCl, 10 mM imidazole), and columns are washed with 100 ml of buffer A. Then, columns are washed with 150 ml of a buffer obtained by mixing 93% of buffer A and 7% of buffer B (50 mM Hepes-HaOH pH7.5, 0.5 m NaCl, 500 mM imidazole). Finally, 60 ml of a buffer obtained by mixing 50% of buffer A and 50% of buffer B is injected into columns. Each 1 ml of the eluted fractions are fractionated, and stored, and an aliquot is analyzed with SDS-PAGE to identify fractions containing 46 Kda of a c-Jun NH₂-terminal kinase.

Further, to increase the activity of NH₂-terminal kinase, the c-Jun NH₂-terminal kinase may be incubated with MKK4/SKK1, active (manufactured by Upstate) and MKK7 beta 1, active (manufactured by Upstate). In this case, a part of kinase assay methods described in manuals annexed to MKK4/SKK1, active (manufactured by Upstate) and MKK7 beta 1, active (manufactured by Upstate) may be modified, and a c-Jun NH₂-terminal kinase can be activated.

The required buffers and reagents include:

(i) Hepes buffer, MgCl₂, ATP:

(ii) Recombinant MKK4/SKK1, active expressed in Escherichia coli (Upstate) stocked at a final concentration of 53.5 μm in 50 mM Tris/HCl, pH 7.5, 150 mM NaCl, 0.1 mM EGTA, 0.03% Brij 35, 270 mM sucrose, 1 mM benzamidine, 0.2 mM PMSF, 0.1% 2-mercaptoethanol; and

(iii) Recombinant MKK7 beta 1, active expressed in Escherichia coli (Upstate) stocked at a final concentration of 14.6 μM in 50 mM Tris/HCl, pH 7.5, 150 mM NaCl, 0.1 mM EGTA, 0.03% Brij 35, 270 mM sucrose, 1 mM benzamidine, 0.2 mM PMSF, 0.1% 2-mercaptoethanol.

Phosphorylation of a purified c-Jun NH₂-terminal kinase is performed at a final volume of 10 ml by the following procedure.

First, all of 1 ml of Milli Q water, 1 ml of a 500 mM Hepes buffer, 2 ml of a mixture of MKK4/SKK1, active (375 nM) and MKK7 beta1, active (375 nM), and 2 ml of a mixture of MgCl₂ (75 mM) and ATP (570 μM) are mixed, and 4 ml of c-Jun NH₂-terminal kinase (1 mg/ml) is added to the mixture. Then, the mixture is incubated at room temperature for 1 hour. All c-Jun NH₂-terminal kinases which have been activated with different batches are pooled, and glycerol is added to this to a final concentration of 10%. Then, this is divided into each 1 ml, and stored at −80° C.

It is to be understood that other methods of activation are also possible, for example; in vivo stimulation as described by In N. Foltz, at al. (The Journal of Biological Chemistry (1998) Vol 273, 9344-9351).

An insect c-Jun NH₂-terminal kinase comprising an amino acid sequence shown in the group B can be used as a research tool. For example, it can be used as a research tool for performing study such as assaying of the pesticidal ability, screening of a chemical substance having a pesticidal ability, and the like. In addition, for example, also in study of analyzing action and mechanism of an agent which acts on a c-Jun NH₂-terminal kinase, a c-Jun NH₂-terminal kinase can be utilized as a research tool.

In addition, polynucleotides encoding amino acid sequences shown in the group B and polynucleotides having a nucleotide sequence having complementarity to them, as well as partial nucleotide sequences of polynucleotides encoding amino acid sequences shown in the group B, or polynucleotides having nucleotide sequences having complementarity to the partial nucleotide sequences, and a polynucleotide complying a nucleotide sequence represented by SEQ ID NO: 3 or 4 can be used as a research tool. For example, a part of them functions as a polynucleotide used in a method of preparing a c-Jun NH₂-terminal kinase as described above. In addition, a part can be used as an important research tool for performing obtaining a polynucleotide shown in a polynucleotide group B using PCR, or obtaining a polynucleotide shown in a polynucleotide group B using hybridization, as described above.

Particularly, upon implementation of screening of a pesticidal agent, they can be used as an experimental tool for an experiment which is performed for screening. Specifically, they can be used as an experimental tool for an experiment which is performed upon implementation of the assaying of a pesticidal ability, screening of a chemical substance having a pesticidal ability, and the like.

Further, the present invention also includes a system which comprises a means to input, store and manage data information of an ability of test substances, wherein said ability is an ability to modulate the activity of an insect c-Jun NH₂-terminal kinase (hereinafter, referred to as means a in some cases), a means to query and retrieve the data information based on a desired criterion (hereinafter, referred to as means b in some cases), and a means to display and output the result which is queried and retrieved (hereinafter, referred to as means c in some cases) (hereinafter, referred to as present system in some cases).

First, a means a will be explained. A means a is a means to, after data information of an ability to modulate the activity of an insect-derived c-Jun NH₂-terminal kinase possessed by the test substance is inputted, store and manage the inputted information, as described above. The information is inputted by an inputting means 1, and is usually memorized in a memory means 2. Examples of an inputting means include means which can input the information such as a keyboard and a mouse. When inputting and storing managing of the information are completed, a procedure progresses to a next means b. For storing, managing the information, a large amount of data may be effectively stored and managed by inputting information having a data structure using a hardware such as a computer, and a software such as OS and database management, and storing the information into a suitable memory device, for example, computer-readable recording medium such as a flexible disc, a photomagnetic disc. CD-ROM, DVD-ROM, and a hard disc.

A means b will be explained. A means b is a means to query and retrieve the data information stored and managed by a means of a based on criterion for obtaining a desired result, as described above. For the information, when criterion for querying and retrieving is inputted by an inputting means 1, and information in conformity with the criterion is selected among the information usually memorized in a memory means 2, a procedure progresses to a next means c. The selected result is usually memorized in a memory means 2 and, further, can be displayed by a displaying outputting means 3.

A means c will be explained. A means c is a means to display and output the result which is queried and retrieved, as described above. Examples of the displaying % outputting means 3 include a display, a printer and the like, and the result may be displayed on a display device of a computer, or may be outputted on a paper by printing.

### EXAMPLES

The present invention will be explained in more detail below by way of Examples, but the present invention is not limited to these particular Examples.

### Example 1

### Extraction of Total RNA from Cotton Aphid and German Cockroach

(1) Extraction of Total RNA from Cotton Aphid.

A population of adults and larvae of cotton aphid (Aphis gossypii), which had been reared on leaves of potted cucumber, was scraped from the surface of the leaves with a small brush, and 630 mg of the obtained population was crushed into a powder in liquid nitrogen using a mortar and a pestle. From the resulting frozen crushed powder, RNA was isolated using a RNA extracting reagent ISOGEN (manufactured by Nippon Gene) as follows. After 10 ml of ISOGEN was added to the frozen crushed powder in the mortar, the crushed powder was ground for 10 minutes while kept on ice. After grinding, a fluid sample was transferred to a 15 ml tube with a pipette, and 2 ml of chloroform (manufactured by Wako Pure Chemical Industries, Ltd.) was added thereto. Immediately, the mixture was vigorously shaken for 15 seconds and then left at rest at room temperature for 3 minutes. Then, the resulting mixture was centrifuged at 12,000×g at 4° C. for 15 minutes, and each 5 ml of aqueous layer were transferred to two new tubes. After 5 ml of ISOGEN was added to each tube, the mixture was immediately shaken vigorously for 15 seconds, and left at rest at room temperature for 3 minutes. Then, the resulting mixture was centrifuged at 12,000×g at 4° C. for 15 minutes, and each 10 ml of aqueous layer were transferred to new 50 ml tubes, respectively. Subsequently, 10 ml of isopropanol (manufactured by Wako Pure Chemical Industries, Ltd.) was added to each tube, and the mixture was kept on ice for 30 minutes. The resulting mixture was centrifuged at 12,000×g at 4° C. for 10 minutes to precipitate RNA. After the supernatant was removed, 20 ml of 70% ethanol was added to the residue. The resulting mixture was centrifuged at 10,000×g at 4° C. for 5 minutes. After the supernatant was removed, the precipitate of total RNA was slightly dried and then dissolved in 1 ml of commercially available RNAse-free water (Nacalai Tesque, Inc.). A concentration of the prepared total RNA (calculated from an absorbance at 260 nm) was 6.9 mg/ml.

(2) Extraction of Total RNA from German Cockroach

Adults, nymphs and oothecae of artificially-reared German cockroach (Blattella germanica) were provided as samples. Ten (10) of adult males and 10 of adult females (individuals from each of which ootheca has been removed) were used as an adult sample of 1.1 g, 10 of nymph males and 10 of nymph females were used as a nymph sample of 1.0 g, and 26 oothecae were used as an ootheca sample of 1.0 g. Three kinds of these samples were separately crushed into a powder in liquid nitrogen using separate mortars and pestles. From each of the resulting frozen crushed powders, RNA was isolated using a RNA extracting reagent ISOGEN (manufactured by Nippon Gene) as follows. After 10 ml of ISOGEN was added to the frozen crushed powder in the mortar, the crushed powder was ground for 10 minutes while kept on ice. After grinding, a fluid sample was transferred to a 15 ml tube with a pipette, and 2 ml of chloroform (manufactured by Wako Pure Chemical Industries, Ltd.) was added thereto. Immediately, the mixture was vigorously shaken for 15 seconds and then left at rest at room temperature for 3 minutes. Then, the resulting mixture was centrifuged at 12,000×g at 4° C. for 15 minutes, and each 5 ml of aqueous layer were transferred to two new tubes. After 5 ml of ISOGEN was added to each tube, the mixture was immediately shaken vigorously for 15 seconds, and left at rest at room temperature for 3 minutes. Then, the resulting mixture was centrifuged at 12,000×g at 4° C. for 15 minutes, and each 10 ml of aqueous layer were transferred to new 50 ml tubes, respectively. Subsequently, 10 ml of isopropanol (manufactured by Wako Pure Chemical Industries, Ltd.) was added to each tube, and the mixture was kept on ice for 30 minutes. The resulting mixture was centrifuged at 12,000×g at 4° C. for 10 minutes to precipitate RNA. After the supernatant was removed, 20 ml of 70% ethanol was added to the residue. The resulting mixture was centrifuged at 10,000×g at 4° C. for 5 minutes. After the supernatant was removed, the precipitate of total RNA was slightly dried and then dissolved in 1 ml of commercially available RNase-free water (Nacalai Tesque, Inc.). A concentration of the prepared total RNA (calculated from absorbance at 260 nm) was 1.1 mg/ml in the case of adult-derived total RNA, was 2.5 mg/ml in the case of nymph-derived total RNA, and 1.4 mg/ml in the case of ootheca-derived total RNA.

### Example 2

### Isolation of Cotton Aphid c-Jun NH₂-Terminal Kinase Gene

First-strand cDNA was prepared using total RNA from cotton aphid, random Primers (Invitrogen) and Superscript III (Invitrogen) for RT-PCR according to the manufacturer's procedure of Superscript III.

A full-length cDNA of cotton aphid c-Jun NH₂-terminal kinase was amplified by PCR using an oligonucleotide comprising the nucleotide sequence of SEQ ID NO: 3 and an oligonucleotide comprising the nucleotide sequence of SEQ ID NO: 4, which are primers specific for the gene, and Pfu Turbo (manufactured by Stratagene) according to the manufacturer's procedure. First-strand cDNA, prepared as described above, was used as template. The PCR conditions used were as follows: an initial denaturation at 94° C. for 10 minutes; followed by 30 cycles of PCR, one cycle being 94° C. for 30 seconds, 50° C. for 30 seconds, and 72° C. for 2 minutes; followed by 72° C. for 7 minutes. The resulting PCR products were analyzed and purified by agarose gel electrophoresis to obtain the 1260 bp DNA of interest. The obtained DNA was cloned into the pCR-blunt vector (Invitrogen). The resulting plasmid was named oGA058-59 in pCRblunt. The cloned DNA was sequenced to confirm that a polynucleotide comprising the nucleotide sequence of SEQ ID No: 2 was contained in the cloned DNA. An amino acid sequence presumed from the nucleotide sequence was the amino acid sequence of SEQ ID No: 1.

### Example 3

### Isolation of German Cockroach c-Jun NH₂-Terminal Kinase Gene

For isolation of homologues of c-Jun NH₂-terminal kinase gene from other insect species such as German cockroach (Blatella germanica), degenerate primers are designed using Codehop program (publicly accessible on the website of Blocks Protein Analysis Server operated within the Fred Hutchinson Cancer Research Center at http://blocks.fhcrc.org/blocks/codehop.html), and based on the sequence of the aforementioned cotton aphid-derived c-Jun NH₂-terminal kinase gene and the previously-known nucleotide sequences of Ancylostoma caninum (NCBI accession number AAF00539), Xenopus laevis (BAB91438, BAB85483), Homo sapiens (NP_{—}620448, NP_{—}002744), Mus musculus (BAC31240), Rattus norvegicus (S43969), Gallus gallus (BAA19188), Drosophila melanogaster (AAF52883, AAC47325), Anopheles gambiae (EAA05905) and Aedes albopictus (AAO31950).

Partial sequences of a homologue of c-Jun NH₂-terminal kinase gene in a selected insect species are amplified by a series of PCR using first-strand cDNA derived from the insect species as a template. Herein, the first-strand cDNA as a template is prepared by the aforementioned method using Superscript III. Amplification by PCR is performed using a set of degenerate primers as a forward primer and a reverse primer as well as Amplitaq Gold (manufactured by Applied Biosystems) according to the manufacturer's procedure annexed to the reagent. PCR conditions are 94° C. for 10 minutes; followed by 40 cycles of PCR, one cycle being 94° C. for 30 seconds, 45° C. for 1 minute, and 72° C. for 1 minute per 1 kb of a length of a predicted amplification product; and followed by 72° C. for 7 minutes. The PCR product is analyzed and purified by agarose gel electrophoresis to obtain DNA of interest. Further, the obtained DNA is cloned into the pCR-XL-TOPO vector (manufactured by Invitrogen), and sequenced.

Thus, partial sequence of a c-Jun NH₂-terminal kinase gene of Blatella germanica comprising the nucleotide sequence of SEQ ID NO: 15 was obtained. Amino acid sequence presumed from the partial sequences was the amino acid sequence of SEQ ID NO: 16.

Then, primers specific for the resulting partial sequences of the insect homologue of c-Jun NH₂-terminal kinase gene are designed, and 3′RACE PCR or 5′RACE PCR is performed in order to obtain a full-length sequence of the gene. 3′ and 5′RACE PCRs are performed employing first-strand cDNA prepared from the insect total RNA as a template and using SMART PCR cDNA Synthesis Kit (manufactured by Clontech) according to the manufacturer's instructions annexed to the kit.

In 3′RACE and 5′RACE reactions, universal primer mix (UPM) contained in SMART PCR cDNA Synthesis Kit is used in combination with a forward primer or a reverse primer which is specific for the sequence of interest. PCR conditions are 1 cycle at 94° C. for 10 minutes; followed by 40 cycles of PCR, one cycle being 94° C. for 15 seconds. 63° C. for 15 seconds, and 72° C. for 1 minute per 1 kb of a length of a predicted amplification product; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

When a distinct amplification product is not obtained by the first-round PCR, nested PCR is performed using the first-round PCR product as a template. As primers, NUP primer contained in SMART PCR cDNA Synthesis it is used in combination with a specific forward primer or a specific reverse primer which is designed to bind internal sequence of the first-round PCR product of interest PCR conditions are 1 cycle at 94° C. for 10 minutes; followed by 40 cycles of PCR, one cycle being 94° C. for 15 seconds, 63° C. for 15 seconds, and 72° C. for 2 minutes; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

The above sequencing results reveal 5′-terminal sequence and 3′-terminal sequence, each encoding N-terminal region and C-terminal region of the insect c-Jun NH₂-terminal kinase, respectively.

### Example 4

### Construction of Recombinant Bacmid

(1) Cloning of c-Jun NH₂-Terminal Kinase cDNA into Gene Expression Vector pFastBac (Registered Trademark) HTb

A 1272 bp DNA fragment, obtained by digesting with EcoRI oGA0513-59 in pCRblunt that had been obtained in Example 2, was isolated and purified, and ligated into the EcoRI cloning sites of the gene expression vector pFastBac (Registered Trademark) HTb. Hereinafter, the resulting vector was named oGA058-59 in pFastBacHTb.

Then, PCR was performed to obtain a 663 bp DNA fragment using Pfu Turbo polymerase (Stratagene) and employing oGA058-59 in pCRblunt obtained in Example 2 as a template. The PCR was performed according to the manual annexed to the reagent and, as primers, an oligonucleotide comprising the nucleotide sequence of SEQ ID NO: 5 and an oligonucleotide comprising the nucleotide sequence of SEQ ID NO: 6, which are primers having high specificity, were used. The PCR conditions were as follows: 1 cycle of 95° C. for 5 minutes: followed by 25 cycles of PCR, one cycle being 95° C. for 30 seconds, 50° C. for 30 seconds and 72° C. for 1 minute and 30 seconds; and followed by 1 cycle of 72° C. for 7 minutes. Then, a DNA fragment of interest was obtained by analyzing and purifying the resulting PCR product by agarose gel electrophoresis. Further, the obtained DNA fragment was cloned into pCR-blunt vector (manufactured by Invitrogen), and a nucleotide sequence of the DNA fragment was determined. The thus obtained vector was designated as oGA060-61 in pCRblunt.

A 605 bp DNA fragment obtained by digesting the above-obtained oGA060-61 in pCRblunt with BamHI/NdeI was isolated and purified, and ligated to the BamHI/NdeI cloning site of oGA058-59 in pFastBacHTb. The resulting vector was designated as pGA01.1. Then, a nucleotide sequence was determined and, as a result, it was found out that a mutation was contained in pFastBacHTb used in construction.

A digestion product obtained by digesting pGA01.1 with BamHI and EcoRI was analyzed and purified by agarose gel electrophoresis to isolate and purify a 1241 bp DNA fragment of interest, and the DNA fragment was cloned into a 4850 bp BamHI/EcoRI fragment of the expression vector pFastBac (Registered Trademark) HTb. The resulting vector was designated pGAO1 and replaced the former pGAO1.1 construct.

### (2) Generation of Recombinant Bacmid DNA

Using the obtained pGAO1, a competent cell of Escherichia coli DH10Bac was transformed. Recombinant bacmid DNA for cotton aphid c-Jun NH₂-terminal kinase was isolated from the transformed Escherichia coli DH10Bac, All procedures were according to the manual annexed to Bac-to-Bac Baculovirus (Trademark) Expression System (manufactured by Invitrogen).

The presence or the absence of the objective gene in the recombinant bacmid was verified by PCR analysis. As the bacmid contains M13 Forward(−40) and M13 Reverse priming sites, the M13 Forward(−40) and the M13 Reverse primers were used. Also a combination the M13 Forward(−40) or M13 Reverse primers and a primer specific for the insert was also used. Each manipulation was performed according to the manual annexed to Bac-to-Bac Baculovirus (Trademark) Expression System (manufactured by Invitrogen). Each PCR condition was as follows.

(a) In the case of an oligonucleotide comprising the nucleotide sequence of SEQ ID NO: 7 and an oligonucleotide comprising the nucleotide sequence of SEQ ID NO: 8:

(i) 94° C. for 5 minutes; followed by

(ii) 40 cycles of 94° C. for 15 seconds, 60° C. for 15 seconds, and 72° C. for 3 minutes; and followed by

(iv) 72° C. for 7 minutes.

(b) In the case of an oligonucleotide comprising the nucleotide sequence of SEQ ID NO: 9 and an oligonucleotide comprising the nucleotide sequence of SEQ ID NO 10:

(i) 94° C. for 5 minutes; followed by

(ii) 16 cycles of 94° C. for 15 seconds, 68° C. (reduction of 0.5° C. every +1 cycle) for 15 seconds, and 72° C. for 3 minutes; followed) by

(iii) 20 cycles of 94° C. for 15 seconds, 60° C. for 15 seconds, and 72° C. for 3 minutes; and followed by

(iv) 72° C., 7 minutes.

(c) In the case of an oligonucleotide comprising the nucleotide sequence of SEQ ID NO: 11 and an oligonucleotide comprising the nucleotide sequence of SEQ ID NO: 12:

(1) 94° C. for 5 minutes; followed by

(ii) 40 cycles of 94° C. for 15 seconds, 60° C. for 15 seconds, and 72° C. for 3 minutes; and followed by

(iv) 72° C. for 7 minutes.

(d) In the case of an oligonucleotide comprising the nucleotide sequence of SEQ ID NO: 8 and an oligonucleotide comprising the nucleotide sequence of SEQ ID NO: 9:

(i) 94° C. for 5 minutes; followed by

(ii) 20 cycles of 94° C. for 15 seconds, 65° C. for 15 seconds, and 72° C. for 3 minutes; followed by

(iii) 25 cycles of 94° C. for 15 seconds, 65° C. for 15 seconds, and 72° C. for 3 minutes (increase of 5 seconds every +1 cycle); and followed by

(iv) 72° C., 7 minutes.

Based on the size of the amplified DNA fragments, two kinds of recombinant bacmids of pgao1.1 and pgao1.8 were selected. DNA fragments amplified from both bacmids had the correct size. Therefore, it was indicated that transposition of the pFastBac (Registered Trademark) expression construct into the bacmid DNA had occurred.

### Example 5

### Preparation of Recombinant Bacmid Stock

### (1) Transfection of Recombinant Bacmid

The recombinant bacmid pgaol.1 was transfected into Sf9 cells (ATCC: CRL-1711) in order to generate recombinant baculovirus according to the manual annexed to Bac-to-Bac Baculovirus (Trademark) Expression System (manufactured by Invitrogen). For the transfection, following materials were used: cellfectin (manufactured by Invitrogen), Grace's Insect Cell Culture Medium supplemented with L-amino acids (manufactured by Invitrogen, Gibco), 10% of Foetal Bovine Serum (manufactured by Clontech), and penicillin/streptomycin (manufactured by Life Technologies). 2 μg of bacmid DNA and 7 μl of cellfectin were used. P1 recombinant baculovirus stock was harvested after 8 days according to the manual annexed to Bac-to-Bac Baculovirus (Trademark) Expression System (manufactured by Invitrogen).

### <Large Scale Preparation of Recombinant Baculovirus>

The cotton aphid c-Jun NH₂-terminal kinase baculovirus stock was amplified by inoculating 0.5 ml of P1 virus stock or subsequent generated virus stock (MOI=0.1) on 30 ml of Sf9 insect cell cultures of 1×10⁶ cells/ml. The amplified baculovirus stocks were harvested after 4 days of culture according to the manual annexed to Bac-to-Bac Baculovirus (Trademark) Expression System (manufactured by Invitrogen). The Sf9 cell cultures were grown as suspension in Erlenmeyers (Elscolab) at 135 rpm and at 27° C. The medium components were as follows: Grace's Insect Cell Culture Medium supplemented with L-amino acids (manufactured by Invitrogen, Gibco), 10% Foetal Bovine Serum (manufactured by Clontech), penicillin/streptomycin (manufactured by Life Technologies), and pluronic F-68 solution at final concentration of 0.1% (manufactured by Sigma-aldrich)

The titer of the baculovirus stock was determined by plaque assay according to the manual annexed to Bac-to-Bac Baculovirus (Trademark) Expression System (manufactured by Invitrogen), but for the medium of the plague assay, 2% agarose was used instead of 4% agarose.

### Example 6

### Expression of c-Jun NH₂-Terminal Kinase in Insect Cell

Sf9 cells of 4×10⁶ were suspended in 30 ml of recombinant baculovirus stock in which the cotton aphid c-Jun NH₂-terminal kinase gene was introduced, and rotation-cultured in a 125 ml Erlenmeyer (manufactured by Elscolab) at 27° C., 135 rpm for 1 hour. Thereafter, the cell suspension was equally distributed over three 250 ml Erlenmeyer (manufactured by Elscolab), and the cell culture medium described in Example 5 was added until a volume of a culturing solution in each Erlenmeyer became 100 ml. The Sf9 cells were rotation-cultured at 135 rpm at 27° C. for 48 hours in the prepared culturing Solution. The cultured solution was centrifuged at 1200 rpm to harvest Sf9 cells infected with baculovirus. The harvested Sf9 cells were flash frozen in liquid nitrogen and stored at −80° C. until further use.

### Example 7

### Purification of c-Jun NH₂-Terminal Kinase

### (1) Preparation of Crude Extract

The frozen Sf9 cells infected with baculovirus were re-suspended in 30 ml of buffer A (50 mM Hepes pH 7.5, 0.5 m NaCl, 10 mM imidazole), and subsequently lysed in buffer A by means of a French Press (manufactured by Thermo Spectronic). The pressure was maintained at 1300-1500 psi during the procedure of breaking of the cells. The French pressed solution was centrifuged at 13,000×g at 2° C. for 20 minutes to obtain supernatant. The obtained supernatant was filtered through a 0.45 μm filter and kept on ice.

### (2) Purification

The aphid c-Jun NH₂-terminal kinase was cloned in frame with the 6×His tag in pFastBacHTb. The recombinant protein has been purified using metal affinity chromatography, using either the HiTrap Chelating HP (Amersham biosciences) or HisTrap HP (Amersham biosciences) columns, according to the instructions of the manufacturer (Amersham biosciences). The purification procedure was undertaken on the AKTA-FPLC (Amersham biosciences).

The HiTrap/HisTrap affinity columns have been prepared according to the manufacturer's protocol (Amersham biosciences). Buffer A, the binding buffer, was made of 50 mM Hepes pH 7.5, 0.5M NaCl, 10 mM imidazole; and buffer B, the elution buffer, was made of 50 mM Hepes pH 7.5, 0.5M NaCl, 500 mM Imidazole. The purification protocol to purify the aphid c-Jun NH₂-terminal kinase included the following steps:

(i) sample injection,

(ii) washing out unbound protein with 10 column volumes (CV) of buffer A,

(iii) washing for 15 CV with 15% buffer B (75 mM imidazole) for Equilibration of the column,

(iv) elution of purified protein with 13 CV 50% buffer B

(250 mM imidazole), and

(v) washing the column with 10 CV 100% buffer B (500 mM imidazole).

The obtained elution fractions were analysed to verify presence of the recombinant, cotton aphid peptidy-dipeptidase A by means of standard techniques of SDS-PAGE and western blot. An 8% polyacrylamide gel was used for optimal gel electrophoresis resolution of the c-Jun NH₂-terminal kinase protein, since the molecular weight of the c-Jun NH₂-terminal kinase protein was 46 kDa. For western blot analysis, an anti-His (H15) sc-803 rabbit polyclonal IgG antibody (manufactured by tebubio) was used as primary antibody at a 1:500 dilution. The secondary antibody was a goat anti-rabbit-HRP (manufactured by Pierce) at a dilution of 1:10000.

After analysis of the gels by SDS-PAGE and Western blotting, the fractions of interest were pooled and glycerol was added to a final concentration of 10%. The protein concentration was determined with the Bradford spectra-photometric protocol using Pre-diluted Protein Assay Standards Bovine Serum Albumin Fraction V Set according to the manufacturer's protocol (Bio-Rad). The pooled fractions were then distributed into several aliquots and immediately flash-frozen in liquid nitrogen and stored at −80° C.

(3) Activation of c-Jun NH₂-Terminal Kinase

To increase the activity of c-Jun NH₂-terminal kinase, the c-Jun NH₂-terminal kinase was incubated with MKK4/SKK1, active (manufactured by Upstate) and MKK7 beta 1, active (manufactured by Upstate). In this case, a part of kinase assay methods described in manuals annexed to MKK4/SKK1, active (manufactured by Upstate) and MKK7 beta 1, active (manufactured by Upstate) was modified, and a c-Jun NH₂-terminal kinase was activated.

The required buffers and reagents were as follows:

(i) Hepes buffer, MgCl₂, ATP;

(ii) Recombinant MKK4/SKK1, active expressed in Escherichia coli (Upstate) stocked at a final concentration of 53.5 μM in 50 mm Tris/HCl, pH 7.5, 150 mM NaCl, 0.1 mM EGTA, 0.03% Brij 35, 270 mM sucrose, 1 mM benzamidine, 0.2 mM PMSF, 0.1% 2-mercaptoethanol; and

(iii) Recombinant MKK7 beta 1, active expressed in Escherichia coli (Upstate) stocked at a final concentration of 14.6 μM in 50 mM Tris/HCl, pH 7.5, 150 mM NaCl, 0.1 mM EGTA, 0.03% Brij 35, 270 mM sucrose, 1 mM benzamidine, 0.2 mM PMSF, 0.1% 2-mercaptoethanol.

Phosphorylation of a purified c-Jun NH₂-terminal kinase was performed at a final volume of 10 ml by the following procedure.

First, all of 1 ml of Milli Q water, 1 ml of a 500 mM Hepes buffer, 2 ml of a mixture of MKK4/SKK1, active (375 nM) and MKK7 betel, active (375 nM), and 2 ml of a mixture of MgCl₂ (75 mM) and ATP (570 μM) were mixed, and 4 ml of c-Jun NH₂-terminal kinase (1 mg/ml) was added to the mixture. Then, the mixture was incubated at room temperature for 1 hour. All c-Jun NH₂-terminal kinases which had been activated with different batches were pooled, and glycerol was added to this to a final concentration of 10%. Then, this was divided into each 1 ml, and stored at −80° C.

### Example 8

### Selection of Compound which Modulates c-Jun NH₂-Terminal Kinase Activity

Selection of a compound which modulates the activity of a c-Jun NH₂-terminal kinase was performed in a system where the activity of a c-Jun NH₂-terminal kinase which is modulated by adding a test compound to an in vitro reaction system using the aphid c-Jun NH₂-terminal kinase prepared in Example 7 is measured and evaluated.

Measurement of the activity of the aphid c-Jun NH₂-terminal kinase was performed using IMAP screening Express Kit with progressive Binding system (manufactured by Molecular Device) according to a method described in the manual annexed to the system and employing, as a substrate, a peptide comprising the amino acid sequence of SEQ ID NO: 13 to which biotin was added at the amino-terminus, and a peptide comprising the amino acid sequence of SEQ ID NO: 14 in which the carboxyl-terminus was fluorescently labeled.

For measuring the activity, the activity of the aphid c-Jun NH₂-terminal kinase was measured when a test compound dissolved in DMSO was contained to a final concentration of 10 μM. In addition, the activity of the aphid c-Jun NH₂-terminal kinase was measured when DMSO was contained in place of a test compound. Then, a ratio (%) of a measured value of the activity of the aphid c-Jun NH₂-terminal kinase when a test compound dissolved in DMSO was contained, relative to a measured value of the activity of aphid c-Jun NH₂-terminal kinase when DMSO was contained in place of the test compound was calculated, and a value obtained by subtracting the calculated value from 100% was adopted as an inhibition degree (%). Results in each test compound are shown in Table 4 in Example 9 together with results of Example 9.

The activity of aphid c-Jun NH₂-terminal kinase was measured when a test compound dissolved in DMSO was contained to a final concentration of each concentration of 100 μM, 30 μM, 10 μM, 3 μM, 1 μM, 0.3 μM, 0.1 μM or 0.03 μM. IC₅₀ (μM) was calculated from the result of each concentration at each test compound using a concentration-dependent test analyzing software XL fit (manufactured by idbs). Results are shown in Table 5 in Example 10 together with results of Example 9.

### Example 9

### Pesticidal Activity Test

A sterilized artificial feed having the following composition (Table 3) was prepared. Then, according to the same manner as that of the method described in Handbook of Insect Rearing Vol. 1 (Elsevier Science Publishers 1985) pp. 35 to pp. 36 except that a test compound dissolved in DMSO to a final concentration of 640 μM was added at 0.5 volume of the artificial feed, and components were mixed, Aphis gossypii was reared. Six days after rearing, the number of surviving Aphis gossypii was investigated, and an entity exhibiting a significant controlling value (e.g. controlling value of 30% or more) was determined to have pesticidal activity by obtaining a controlling value by the following equation.

Controlling value(%)={1−(Cb×Tai)/(Cai×Tb)}×100

Letters in the equation represent the following meanings.

Cb: Number of surviving worms before treatment in non-treated section
Cai: Number of surviving worms at observation in non-treated section
Tb: Number of surviving worms before treatment in treated section
Tai: Number of surviving worms at observation in treated section

Results are shown in Table 4 in Example 9 together with results of Example 8.

TABLE 3

| | (mg/100 ml) |
|---|---|
| Amino acid | |
| L-alanine | 100.0 |
| L-arginine | 275.0 |
| L-asparagine | 550.0 |
| L-aspartic acid | 140.0 |
| L-cysteine | 40.0 |
| (hydrochloride) | |
| L-glutamic acid | 140.0 |
| L-glutamine | 150.0 |
| L-glycine | 80.0 |
| L-histidine | 80.0 |
| L-isoleucine | 80.0 |
| L-leucine | 80.0 |
| L-lysine | 120.0 |
| (hydrochloride) | |
| L-methionine | 80.0 |
| L-phenylalanine | 40.0 |
| L-proline | 80.0 |
| L-serine | 80.0 |
| L-threonine | 140.0 |
| L-tryptophan | 9 80.0 |
| L-tyrosine | 40.0 |
| L-valine | 80.0 |
| Vitamins | |
| Ascorbic acid | 100.0 |
| Biotin | 0.1 |
| Calcium | 5.0 |
| pantothenate | |
| Choline chloride | 50.0 |
| Inositol | 50.0 |
| Nicotinic acid | 10.0 |
| Thiamine | 2.5 |
| Others | |
| Sucrose | 12500.0 |
| Dipotassium | 1500.0 |
| hydrogen phosphate | |
| Magnesium sulfate | 123.0 |
| Cupric chloride | 0.2 |
| Ferric chloride | 11.0 |
| Manganese chloride | 0.4 |
| Zinc sulfate | 0.8 |
| (anhydrous) | |

Adjusted to pH 6.8

TABLE 4

| | | Activity of | |
|---|---|---|---|
| | | inhibiting c-Jun | |
| | | NH₂-terminal | Determination |
| | | kinase activity | result of |
| | | (inhibition degree (%) | pesticidal |
| Compound name | Structure | at 10 μM addition) | activity |
| Anthra[1,9-CD] pyrazole-6(2H)-one | 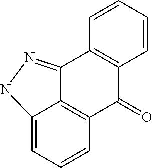 | 83 | Presence of pesticidal activity |
| Arcyriarubin A | 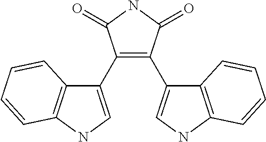 | 73 | Presence of pesticidal activity |
| 4-amino-1-tert- butyl-3-(1-naphthyl) pyrazolo(3,4-d) pyrimidine | 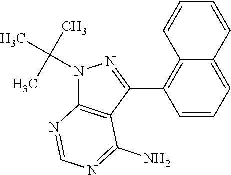 | 68 | Presence of pesticidal activity |

### Example 10

### Pesticidal Activity Test

According to the same manner as that of Example 9 except that a test compound dissolved in DMSO to a final concentration of 50 ppm was added, pesticidal activity test was performed, and results are shown in Table 5 in Example 10 together with results of Example 8.

TABLE 5

| | | Activity of | |
|---|---|---|---|
| | | inhibiting c-Jun | Determination |
| | | NH₂-terminal | result of |
| Compound | | kinase activity | pesticidal |
| name | Structure | (IC50, (μM)) | activity |
| PH09940 | 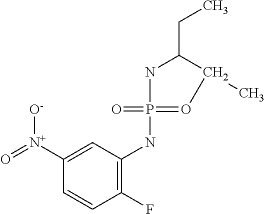 | 35.21 | Presence of pesticidal activity |
| DE01948 | 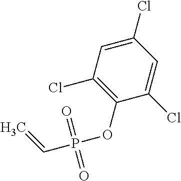 | 21.18 | Presence of pesticidal activity |
| DF06524 | 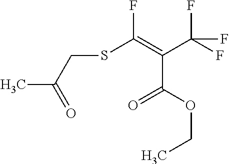 | 47.86 | Presence of pesticidal activity |
| DB04029 | 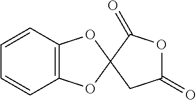 | >100 | Absence of pesticidal activity |
| DC05429 | 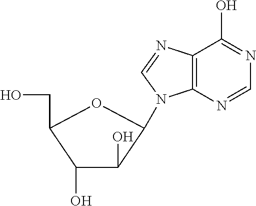 | >100 | Absence of pesticidal activity |
| DD08068 | 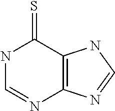 | >100 | Absence of pesticidal activity |
| DD08100 | 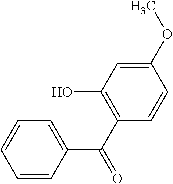 | >100 | Absence of pesticidal activity |

### INDUSTRIAL APPLICABILITY

According to the present Invention, it becomes possible to provide a more target-based approach of screening agricultural chemicals, whereby compounds are screened against a specific target that has been identified as biologically and/or physiologically relevant with intent of chemically interfering with the target site to control insects or other pest organisms.

### Free Text in Sequence Listing

### SEQ ID NO: 3

Designed oligonucleotide primer for PCR

### SEQ ID NO: 4

Designed oligonucleotide primer for PCR

### SEQ ID NO: 5

Designed oligonucleotide primer for PCR

### SEQ ID NO: 6

Designed oligonucleotide primer for PCR

### SEQ ID NO: 7

Designed oligonucleotide primer for PCR

### SEQ ID NO: 8

Designed oligonucleotide primer for PCR

### SEQ ID NO: 9

Designed oligonucleotide primer for PCR

### SEQ ID NO: 10

Designed oligonucleotide primer for PCR

### SEQ ID NO: 11

Designed oligonucleotide primer for PCR

### SEQ ID NO: 12

Designed oligonucleotide primer for PCR

### SEQ ID NO: 13

Designed peptide for JNK substrate

### SEQ ID NO: 14

Designed peptide for JNK substrate

1. An agent that modulates physiological condition of pests, wherein said agent has an ability to modulate the activity of an insect c-Jun NH₂-terminal kinase.

2. An agent according to claim 1, wherein said c-Jun NH₂-terminal kinase is a cotton aphid c-Jun NH₂-terminal kinase.

3. An agent according to claim 1, wherein said agent is a pesticidal agent.

4. An agent according to claim 1, wherein said ability to modulate the activity of an insect c-Jun NH₂-terminal kinase is an ability to inhibit a reaction of the insect c-Jun NH₂-terminal kinase with the peptide of SEQ ID NO: 14.

5. A pesticidal agent which comprises a substance that has an ability to modulate the activity of an insect c-Jun NH₂-terminal kinase or an agriculturally acceptable salt of the substance as an active ingredient.

6. A pesticidal agent according to claim 5, wherein said substance has an ability to inhibit a reaction of the insect c-Jun NH₂-terminal kinase with the peptide of SEQ ID NO: 14.

7. A pesticidal agent according to claim 6, wherein said substance has an ability to inhibit the reaction of the insect c-Jun NH₂-terminal kinase with the peptide of SEQ ID NO: 14 in a cell-free system, wherein in the presence of said substance of 10 micro M or more the activity of said c-Jun NH₂-terminal kinase is lower than that in the absence of said substance.

8. A pesticidal agent according to claim 6, wherein said substance has an ability to inhibit a reaction of the insect c-Jun NH₂-terminal kinase with the peptide of SEQ ID NO: 14 in a cell-free system with an IC₅₀ of 100 micro M or less.

9. A method for assaying pesticidal activity of a test substance, which comprises:
(1) a first step of measuring the activity of a c-Jun NH₂-terminal kinase selected from the following group A in a reaction system in which said c-Jun NH₂-terminal kinase contacts with a test substance; and
(2) a second step of evaluating the pesticidal activity of said test substance based on the difference obtained by comparing the activity measured in the first step with the activity of a control:
<group A>
(a) a protein comprising the amino acid sequence of SEQ ID NO: 1;
(b) a protein comprising an amino acid sequence with deletion, addition or substitution of one or more amino acids in the amino acid sequence of SEQ ID NO: 1, wherein said protein has c-Jun NH₂-terminal kinase activity;
(c) a protein comprising an amino acid sequence that has sequence identity of 83% or more to the amino acid sequence of SEQ ID NO: 1, wherein said protein has c-Jun NH₂-terminal kinase activity:
(d) a protein comprising the amino acid sequence encoded by the nucleotide sequence of SEQ ID NO: 2;
(e) a protein comprising an amino acid sequence encoded by a nucleotide sequence that has sequence identity of 65% or more to the nucleotide sequence of SEQ ID NO: 2, wherein said protein has c-Jun NH₂-terminal kinase activity;
a protein comprising an amino acid sequence encoded by a polynucleotide, wherein said polynucleotide hybridizes under a stringent condition to a polynucleotide comprising a nucleotide sequence complementary to the nucleotide sequence of SEQ ID NO: 2, and wherein said protein has c-Jun NH₂-terminal kinase activity;
(g) a protein comprising an amino acid sequence of an insect c-Jun NH₂-terminal kinase; and
(h) a protein comprising an amino acid sequence of a cotton aphid c-Jun NH₂-terminal kinase.

10. A method for screening a pesticidal substance, which comprises selecting a substance having the pesticidal activity that is evaluated by the method according to claim 9.

11. A pesticidal agent which comprises a substance selected by the method according to claim 10 or agriculturally acceptable salts thereof as an active ingredient.

12. A method for controlling pests which comprises applying an effective amount of the pesticidal agent according to claim 5, 6, 7, 8 or 11 to the pest, habitat of the pest or plant to be protected from the pest.

13. A method for controlling pests which comprises:
identifying a substance having the pesticidal activity that is evaluated by the method according to claim 9, and
contacting the pest with the identified pesticidal substance.

14. An insect c-Jun NH₂-terminal kinase comprising an amino acid sequence selected from the following group B:
<group B>
(a) the amino acid sequence of SEQ ID NO: 1;
(b) an amino acid sequence with deletion, addition or substitution of one or more amino acids in the amino acid sequence of SEQ ID NO: 1, wherein said amino acid sequence has c-Jun NH₂-terminal kinase activity;
(c) an amino acid sequence that has sequence identity of 95% or more to the amino acid sequence of SEQ ID NO: 1, wherein said amino acid sequence has c-Jun NH₂-terminal kinase activity;
(d) the amino acid sequence encoded by the nucleotide sequence of SEQ ID NO: 2;
(e) an amino acid sequence encoded by a nucleotide sequence that has sequence identity of 75% or more to the nucleotide sequence of SEQ ID NO: 2, wherein said amino acid sequence has c-Jun NH₂-terminal kinase activity;
(f) an amino acid sequence encoded by a polynucleotide, wherein said polynucleotide hybridizes under a stringent condition to a polynucleotide comprising a nucleotide sequence complementary to the nucleotide sequence of SEQ ID NO: 2, wherein said amino acid sequence has c-Jun NH₂-terminal kinase activity; and
(g) an amino acid sequence of a cotton aphid c-Jun NH₂-terminal kinase.

15. Use of an insect c-Jun NH₂-terminal kinase as a reagent that provides an indicator to evaluate pesticidal activity.

16. Use of an insect c-Jun NH₂-terminal kinase according to claim 14 as a reagent that provides an indicator to evaluate pesticidal activity.

17. A polynucleotide which comprises a nucleotide sequence encoding an amino acid sequence of a c-Jun NH₂-terminal kinase according to claim 14

18. A polynucleotide according to claim 17, which comprises the nucleotide sequence of SEQ ID NO: 2.

19. A polynucleotide which comprises a nucleotide sequence complementary to a nucleotide sequence of a polynucleotide according to claim 17 or 18.

20. A polynucleotide which comprises:
a partial nucleotide sequence of a polynucleotide according to claim 17 or 18; or
a nucleotide sequence complementary to said partial nucleotide sequence.

21. A polynucleotide according to claim 20, which comprises a nucleotide sequence of SEQ ID NO: 3 or 4.

22. A method for obtaining a polynucleotide comprising a nucleotide sequence encoding an amino acid sequence of a c-Jun NH₂-terminal kinase, which comprises:
a step of amplifying a desired polynucleotide by polymerase chain reaction using as a primer a polynucleotide according to claim 20;
a step of identifying the desired polynucleotide amplified; and
a step of recovering the identified polynucleotide.

23. A method for obtaining a polynucleotide comprising a nucleotide sequence encoding an amino acid sequence of a c-Jun NH₂-terminal kinase, which comprises:
a step of detecting a desired polynucleotide by hybridization using as a probe a polynucleotide according to claim 19;
a step of identifying the desired polynucleotide detected; and
a step of recovering the identified polynucleotide.

24. A circular polynucleotide comprising a nucleotide sequence of a polynucleotide according to claim 17 or 18, wherein said nucleotide sequence is operably linked to a baculovirus promoter.

25. A circular polynucleotide according to claim 24, wherein said promoter is a polyhedrin gene promoter.

26. A circular polynucleotide according to claim 24 or 25, wherein said polynucleotide comprises a replication origin for autonomous replication in a host cell.

27. A circular polynucleotide according to claim 24, wherein said polynucleotide comprises a nucleotide sequence of a baculovirus shuttle vector and capable of propagating as a virus in an insect cell.

28. A method for producing a circular polynucleotide, which comprises ligating a polynucleotide according to claim 17 or 18 into a vector.

29. A transformant in which a polynucleotide according to claim 17 or 18 is introduced.

30. A transformant according to claim 29, wherein said transformant is a transformed insect cell.

31. A method for producing a transformant, which comprises introducing a polynucleotide according to claim 17 or 18 into a host cell.

32. A recombinant baculovirus comprising within its genome a polynucleotide according to claim 17 or 18.

33. A method for producing a c-Jun NH₂-terminal kinase, which comprises a step of culturing the transformant according to claim 29 and recovering a produced c-Jun NH₂-terminal kinase.

34. Use of a c-Jun NH₂-terminal kinase comprising an amino acid sequence selected from the following group B:
<group B>
(a) the amino acid sequence of SEQ ID NO: 1;
(b) an amino acid sequence with deletion, addition or substitution of one or more amino acids in the amino acid sequence of SEQ ID NO: 1, wherein said amino acid sequence has c-Jun NH₂-terminal kinase activity;
(c) an amino acid sequence that has sequence identity of 95% or more to the amino acid sequence of SEQ ID NO: 1, wherein said amino acid sequence has c-Jun NH₂-terminal kinase activity;
(d) the amino acid sequence encoded by the nucleotide sequence of SEQ ID NO: 2;
(e) an amino acid sequence encoded by a nucleotide sequence that has sequence identity of 75% or more to the nucleotide sequence of SEQ ID NO: 2, wherein said amino acid sequence has c-Jun NH₂-terminal kinase activity;
(f) an amino acid sequence encoded by a polynucleotide, wherein said polynucleotide hybridizes under a stringent condition to a polynucleotide comprising a nucleotide sequence complementary to the nucleotide sequence of SEQ ID NO: 2, wherein said amino acid sequence has c-Jun NH₂-terminal kinase activity; and
(g) an amino acid sequence of a cotton aphid c-Jun NH₂-terminal kinase or a polynucleotide according to claim 17 as a research tool.

35. Use according to claim 34, wherein the research tool is an experimental tool for screening a pesticidal substance.

36. A system which comprises:
a means to input, store and manage a data information of an ability of test substances, wherein said ability is an ability to modulate the activity of an insect c-Jun NH₂-terminal kinase;
a means to query and retrieve the data information based on a desired criterion; and
a means to display and output the result which is queried and retrieved.
